(19) United States
(12) Patent Application Publication
(10) Pub. No.: US 2023/0149523 A1
(43) Pub. Date: May 18, 2023

(54) TREATMENT OF AUTOIMMUNITY AND TRANSPLANT REJECTION THROUGH ESTABLISHMENT AND/OR PROMOTION OF TOLEROGENIC PROCESSES BY FIBROBLAST-MEDIATED REPROGRAMMING OF ANTIGEN PRESENTING CELLS

(71) Applicant: FIGENE, LLC, Houston, TX (US)

(72) Inventor: Thomas ICHIM, San Diego, CA (US)

(21) Appl. No.: 17/754,809

(22) PCT Filed: Oct. 13, 2020
(86) PCT No.: PCT/US2020/055427

§ 371 Date: Apr. 13, 2022

Related U.S. Application Data
(60) Provisional application No. 62/914,747, filed on Oct. 14, 2019.

(51) Int. Cl. A61K 39/00; C12N 5/077; C12N 5/0784; A61K 35/33; A61K 35/28; A61K 38/20

(52) U.S. Cl. CPC A61K 39/0008; A61K 35/28; A61K 35/33; A61K 38/2013; A61K 39/001; C12N 5/0639; C12N 5/0656; C12N 2501/052

(57) ABSTRACT

The disclosure provides means of treating autoimmunity through reprogramming of antigen presenting cells in an individual in need of treatment through administration of fibroblasts and/or derivatives of fibroblasts. In one embodiment, fibroblasts are administered in an allogeneic manner subsequent to modification which endows fibroblast ability to alter antigen presenting cells in a manner which supports the generation of immunological tolerance as opposed to immunological rejection. In one embodiment, fibroblasts are utilized to decrease costimulatory molecule expression on antigen presenting cells, in order to allow for production of antigen-specific immunological tolerance promoting cells.

This application claims priority to U.S. Provisional Pat. Application Serial No. 62/914,747, filed Oct. 14, 2019, which is incorporated by reference herein in its entirety.

### TECHNICAL FIELD

Embodiments of the disclosure include at least the fields of molecular biology, cell biology, immunology, and medicine.

### BACKGROUND

The utilization of immunotherapy as a medical intervention has led to significant improvements in the area of oncology, where the immune system is treated to selectively “seek and destroy” neoplastically-transformed cells. The advent of checkpoint inhibitors, which selectively suppress molecules that are known to restrain immunity, has allowed for up to 30% remissions in patients previously believed to be untreatable.

Despite these advances in the area of cancer, the utilization of immunotherapy in the area of autoimmunity has been lagging. Previous means of treating autoimmunity include approaches that seek to either globally suppress the immune system, or selectively block the autoreactive immunocyte clones. Examples of approaches that globally suppress the immune system include drugs such as cyclosporin, which blocks all T cells from activation through suppression of the calcineurin pathway. Somewhat more selective inhibitors include agents such as rituximab, which block only B cells, thus allowing some other components of the immune system to remain intact such as T cells, NK cells, and granulocytes. Unfortunately, most of the antigen-nonspecific means of blocking immunity possess the disadvantages of suppressing immune responses that otherwise may be beneficial. Indeed various infections and cancers have been associated with some of the non-specific immune suppressants.

Antigen specific modulation of immunity has been the Holy Grail of immunologists for more than a century. Conceptually, induction of tolerance should be relatively simple; administer the antigen to which tolerance is desired, in absence of a “danger signal”. Essentially, the immune system is preprogrammed to delete autoreactive cells in the thymus. This process, termed “thymic selection” ensures that the majority of T cells that are produced are reactive towards everything else that is not thymus. Interesting studies have demonstrated that thymic medullary epithelial cells express an enzyme called AIRE, which promotes transcriptional promiscuity and thus allows essentially all antigens that the person will express throughout their lifetime to be expressed in the neonatal thymus.

Autoreactive T cells that escape thymic deletion are either killed, or enter a state of anergy if they encounter an autoantigen in absence of costimulatory signals. Costimulatory signals are molecules generated by cells of the innate immune system subsequent to exposure to “danger”. This explains why in many situations autoimmunity is initiated by exposure to viral or bacterial agents. These agents serve as a “danger” signal, which induce expression of costimulatory molecules, which then allow for activation and expansion of autoreactive T cells. The same concept holds true in cancer. In cancer patients, T cells do not recognize the tumors, however, in the presence of danger signals, such as bacterial or viral infections, the T cells become activated, and as a result, in some patients, tumor regression occurs.

Various techniques that have been successfully utilized in animal models have been attempted clinically with futile results. Approaches such as antigen-specific immunization, oral tolerance, intravenous tolerance, and cell therapy induced tolerance have all give mediocre results to date.

The current disclosure provides, in certain embodiments, new ways of expanding induction of immunological tolerance through the utilization of fibroblasts as a cellular adjuvant to induce the generation of antigen-specific tolerogenic mechanisms.

### BRIEF SUMMARY

The present disclosure is directed to systems and methods and compositions for inhibiting and/or treating a pathological immune response. The present disclosure is also directed to systems and methods and compositions for inducing immune tolerance in an individual or for cells of an individual. The pathological immune response may comprise at least one autoimmune reaction, autoimmune disease, graft rejection, graft versus host disease, host versus graft disease, or a combination thereof.

Certain embodiments concern methods for inhibiting and/or treating a pathological immune response and/or inducing immune tolerance, comprising cell to cell contact and/or transfer of soluble materials from a first cell or cells to a second cell or cells. In some embodiments, the cell to cell contact and/or transfer of soluble materials occurs in vivo in an individual in which the first cells or cells may be administered to the individual. In some embodiments, the cell to cell contact and/or transfer of soluble materials occurs in vitro or ex vivo. In some embodiments, the first cell(s) comprise(s) fibroblasts and/or mesenchymal stem cells. In some embodiments, the second cell(s) comprise(s) one or more antigen presenting cells. The antigen presenting cells may be any cells that sufficiently present antigen, such as to activate cytotoxic or tolerogenic immune cells. The antigen presenting cells may comprise, for example, dendritic cells, B cells, innate lymphoid cells, or a combination thereof. In some embodiments, the dendritic cells are selected from the group consisting of lymphoid dendritic cells, myeloid dendritic cells, myeloid suppressor cells, and a combination thereof. In some embodiments the innate lymphoid cells are selected from the group consisting of innate lymphoid cells (ILC)1, ILC2, ILC3, lymphoid tissue inducer cells, and a combination thereof.

In some embodiments, the cell to cell contact and/or transfer of soluble materials from a first cell or cells to a second cell or cells reduces antigen presenting cell activity and/or reprograms antigen presenting cells. The antigen presenting cell activity may comprise expression of MHC molecules on the surface of the antigen presenting cell, loading of antigen into MHC molecules, and/or expression of one or more costimulatory molecules on the antigen presenting cells. The costimulatory molecule(s) may be membrane-bound (including CD40, CD80, and/or CD86) and/or soluble (including IL-12, IL-2, IL-11, IL-15, and/or IL-18).

In some embodiments, the fibroblasts and/or mesenchymal stem cells are derived from particular tissue, including tissue selected from the group consisting of placenta, cord blood, mobilized peripheral blood, omentum, hair follicle, skin, bone marrow, adipose tissue, Wharton’s Jelly, and a combination thereof. In some embodiments, the fibroblasts and/or mesenchymal stem cells are from dermis.

In some embodiments, the fibroblasts are pretreated with one or more toll like receptor (TLR) agonists. The fibroblasts may be pretreated with TLR agonist(s) for a sufficient time and at a sufficient concentration to enhance immune modulatory activity. The immune modulatory activity may comprise activity to suppress antigen presenting cell maturation and/or antigen presenting cell activity. The TLR agonist(s) may be selected from the group consisting of a TLR-1 agonist, TLR-2 agonist, TLR-3 agonist, TLR-4 agonist, TLR-5 agonist, TLR-6 agonist, TLR-7 agonist, TLR-8 agonist, TLR-9 agonist, and a combination thereof. The TLR-1 agonist may comprise Pam3CSK4. The TLR-2 agonist may comprise HKLM. The TLR-3 agonist may comprise Poly:IC. The TLR-4 agonist may be selected from the group consisting of lipopolysaccharide (LPS), buprenorphine, carbamazepine, fentanyl, levorphanol, methadone, cocaine, morphine, oxcarbazepine, oxycodone, pethidine, glucuronoxylomannan from Cryptococcus, morphine-3-glucuronide, lipoteichoic acid, (β-defensin 2, small molecular weight hyaluronic acid, fibronectin EDA, snapin, tenascin C, and a combination thereof. The TLR-5 agonist may comprise flagellin. The TLR-6 agonist may comprise FSL-1. The TLR-7 agonist may comprise imiquimod. The TLR-8 agonist may comprise ssRNA40/LyoVec. The TLR-9 agonist may comprise CpG oligonucleotide, ODN2006, agatolimod, or a combination thereof.

Certain embodiments concern combining fibroblasts and mesenchymal stem cells for use in methods encompassed herein. In some embodiments, mesenchymal stem cells are administered with fibroblasts. The mesenchymal stem cells may enhance immune modulatory effects of fibroblasts. The immune modulatory effects may comprise suppression of maturation of antigen presenting cells. The immune modulatory effects may comprise suppression of NF-kappa B activity, IL-2 production, IL-12 production, IL-15 production, IL-18 production, or a combination thereof by the antigen presenting cells.

In some embodiments, T regulatory cell production in an individual is concurrently increased with administration of the fibroblasts to the individual. T regulatory cell production may be increased by the administration of IL-2, such as low dose IL-2. The low dose IL-2 may comprise a dose of IL-2 between 50,000 to 5,000,000 IU per day; 500,000 to 5,000,000 IU per day; 700,000 to 2,000,000 IU per day; or 1,000,000 to 2,000,000 IU per day. The low dose IL-2 may comprise 1,500,000 IU of IL-2 per day. T regulatory cell production may increase and/or enhance tolerogenic process and/or reprogramming of antigen presenting cells towards a tolerogenic phenotype.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 shows fibroblasts and LPS activated fibroblast suppress TNF-induced CD40 expression on DCs. In the groupings of four bars, control is left, TNF is second from left, TNF+fibroblasts is second from right, and TNF+LPS fibroblast is on the right.

FIG. 2 shows fibroblasts and LPS activated fibroblast suppress TNF-induced CD80 expression on DCs. In the groupings of four bars, control is left, TNF is second from left, TNF+fibroblasts is second from right, and TNF+LPS fibroblast is on the right.

FIG. 3 fibroblasts and LPS activated fibroblast suppress TNF-induced CD86 expression on DCs. In the groupings of four bars, control is left, TNF is second from left, TNF+fibroblasts is second from right, and TNF+LPS fibroblast is on the right.

FIG. 4 shows fibroblasts and LPS activated fibroblast suppress TNF-induced IL-12 production from DCs. In the groupings of four bars, control is left, TNF is second from left, TNF+fibroblasts is second from right, and TNF+LPS fibroblast is on the right.

FIG. 5 shows fibroblasts augment production of IL-10 by activated DCs. In the groupings of four bars, control is left, TNF is second from left, TNF+fibroblasts is second from right, and TNF+LPS fibroblast is on the right.

FIG. 6 shows fibroblasts augment production of IL-1 receptor antagonist (RA) by activated DCs. In the groupings of four bars, control is left, TNF is second from left, TNF+fibroblasts is second from right, and TNF+LPS fibroblast is on the right.

FIG. 7 shows fibroblasts augment expression of PD-L1 by activated DCs. In the groupings of four bars, control is left, TNF is second from left, TNF+fibroblasts is second from right, and TNF+LPS fibroblast is on the right.

FIG. 8 shows fibroblasts are superior to MSCs at suppressing CD40 from activated DCs. In the groupings of four bars, fibroblast is left, MSC is second from left, LPS+fibroblasts is second from right, and MSC+LPS is on the right.

FIG. 9 shows fibroblasts are superior to MSCs at suppressing CD80 from activated DCs. In the groupings of four bars, fibroblast is left, MSC is second from left, LPS+fibroblasts is second from right, and MSC+LPS is on the right.

FIG. 10 shows fibroblasts are superior to MSCs at suppressing CD86 from activated DCs. In the groupings of four bars, fibroblast is left, MSC is second from left, LPS+fibroblasts is second from right, and MSC+LPS is on the right.

FIG. 11 shows fibroblasts are superior to MSCs at suppressing IL-12 from activated DCs. In the groupings of four bars, fibroblast is left, MSC is second from left, LPS+fibroblasts is second from right, and MSC+LPS is on the right.

FIG. 12 shows fibroblasts are superior to MSCs at inducing IL-10 production from activated DCs. In the groupings of four bars, fibroblast is left, MSC is second from left, LPS+fibroblasts is second from right, and MSC+LPS is on the right.

FIG. 13 shows fibroblasts are superior to MSCs at inducing IL-1 RA production from activated DCs. In the groupings of four bars, fibroblast is left, MSC is second from left, LPS+fibroblasts is second from right, and MSC+LPS is on the right.

FIG. 14 shows fibroblasts are superior to MSCs at inducing PD-L1 expression from activated DCs. In the groupings of four bars, fibroblast is left, MSC is second from left, LPS+fibroblasts is second from right, and MSC+LPS is on the right.

### DETAILED DESCRIPTION

### I. General Embodiments

Certain embodiments concern methods of inhibiting a pathological immune response, such as a reduction of antigen presenting cell activity and/or reprogramming of antigen presenting cells, in order to allow for a tolerogenic response. The reduction of antigen presenting cell activity and/or reprogramming of antigen presenting cells may be accomplished by cell to cell contact and/or transfer of soluble materials from one or more fibroblasts to at least one antigen presenting cell. In some embodiments, the cell to cell contact and/or transfer of soluble materials from one or more fibroblasts to at least one antigen presenting cell occurs in an individual, such as an individual that has been administered the fibroblasts. In some embodiments, the cell to cell contact and/or transfer of soluble materials from one or more fibroblasts to at least one antigen presenting cell occurs in vitro or ex vivo, including wherein the fibroblasts are co-cultured with at least one antigen presenting cell, such as an antigen presenting cell from any individual encompassed herein.

In some embodiments, the pathological immune response is an autoimmune response, such as an autoimmune response in an individual. The autoimmune response may be associated with, result in, and/or cause the production of inflammatory cytokines, including inflammatory cytokines selected from the group consisting of interleukin-1, interleukin-6, interleukin-7, interleukin-8, interleukin-9, interleukin-12, interleukin 15, interleukin-17, interleukin-18, interleukin-22, interleukin-23, interleukin-27, TNF-alpha, TNF-beta, interferon alpha, interferon beta, interferon gamma, and a combination thereof. The autoimmune response may be associated with, result in, and/or cause the production of inflammatory markers, including inflammatory markers selected from the group consisting of Apo A1 (Apolipoprotein A1), Beta-2 Microglobulin, Clusterin, CRP (C Reactive Protein), Cystatin-C, Eotaxin, Factor VII, FGF-9 (Fibroblast Growth Factor-9), GCP-2 (Granulocyte Chemotactic Protein-2), Growth Hormone, IgA (Immunoglobulin A), Insulin, IP-10 (Inducible Protein-10), Leptin, LIF (Leukemia Inhibitory Factor), MDC (Macrophage-Derived Chemokine), MIP-1alpha (Macrophage Inflammatory Protein-1alpha), MIP-1beta (Macrophage Inflammatory Protein-1beta), MIP-1gamma (Macrophage Inflammatory Protein-1gamma), MIP-2 (Macrophage Inflammatory Protein-2), MIP-3beta (Macrophage Inflammatory Protein-3beta), MPO (Myeloperoxidase), Myoglobin, NGAL (Lipocalin-2), OSM (Oncostatin M), Osteopontin, SAP (Serum Amyloid P), SCF (Stem Cell Factor), SGOT (Serum Glutamic-Oxaloacetic Transaminase), TIMP-1 (Tissue Inhibitor of Metalloproteinase Type-1), Tissue Factor, TPO (Thrombopoietin) and VEGF (Vascular Endothelial Cell Growth Factor), and a combination thereof.

The antigen presenting cell(s) encompassed herein may be any cell(s) that sufficiently presents antigen, such as a dendritic cell (DC) (including a lymphoid DC, myeloid DC, and/or myeloid suppressor cell), a B cell, and/or an innate lymphoid like cell (ILC) (including ILC1, ILC2, ILC3, and/or lymphoid tissue inducer cell). The dendritic cell, including lymphoid DC, myeloid DC, and/or myeloid suppressor cell, may express DEC-205 and/or CD56. The B cell(s) may express CD5 and/or CD10 and may produce IL-10. The ILC1 cell may express T bet and respond to IL-12 by secretion of IFNγ and may lack expression of CD56. The ILC2 cell may produce IL-4 and/or IL-13. The ILC3 cell may produce IL-17a and/or IL-22. The lymphoid tissue inducer cell may be involved in the induction of memory T cells.

The antigen presenting cell(s) may activate, or be capable of activating, one or more Th1 cells, such as Th1 cells that secrete, or are capable of secreting, cytokines, including cytokines selected from the group consisting of IFNy, IL-2, TNFβ, IL-15, IL-18, IL27, and a combination thereof. The Th1 cells encompassed herein may express, or be capable of expressing, markers selected from the group consisting of CD4, CD94, CD119 (IFNγ R1), CD183 (CXCR3), CD186 (CXCR6), CD191 (CCR1), CD195 (CCR5), CD212 (IL-12Rβ1&2), CD254 (RANKL), CD278 (ICOS), IL-18R,MRP1, NOTCH3, TIM3, and a combination thereof. The Th1 cells may induce, or possess an ability to induce, damage to tissue, including tissue in an individual, through the production of inflammatory cytokines. The inflammatory cytokines may be produced by bystander cells of the immune system.

The antigen presenting cell(s) may activate, or be capable of activating, Th2 cells, such as in a contact-dependent manner. The Th2 cells may secrete, or be capable of secreting cytokines, including cytokines selected from the group consisting of IL-4, IL-5, IL-6, IL-9, IL-10, IL-13, and a combination thereof. The Th1 cells encompassed herein may express, or be capable of expressing, markers selected from the group consisting of CRTH2, CCR4, CCR3, and a combination thereof.

The antigen presenting cell(s) may activate, or be capable of activating, differentiation, proliferation, and/or cytokine production in Th17 cells. The Th17 cells may produce, or be capable of producing, IL-17 and/or IL-22, either constitutively or inducibly. The Th17 cells encompassed herein may express, or be capable of expressing, markers selected from the group consisting of IL-23 receptor, RORyT, CD200, BTLA, IL-18 receptor, CD99, IL-1 receptor 1, CCR4, CCR6, CD26, and a combination thereof.

The antigen presenting cell activity may comprise expression of MHC molecules on the surface of the antigen presenting cell. The reduction of antigen presenting cell activity may comprise the reduction of MHC molecules on the surface of the antigen presenting cell, including reduction of expression of MHC molecules. The antigen presenting cell activity may comprise loading of antigen into MHC molecules on the surface of the antigen presenting cell. The reduction of antigen presenting cell activity may comprise the reduction of loading of antigen into MHC molecules on the surface of the antigen presenting cell, including reduction of loading of antigen into MHC molecules. The antigen presenting cell activity may comprise expression of co-stimulatory molecules in the antigen presenting cell. The reduction of antigen presenting cell activity may comprise the reduction of co-stimulatory molecules in antigen presenting cell, including reduction of expression of co-stimulatory molecules. The co-stimulatory molecules may be membrane bound (such as CD40, CD80, CD86) and/or soluble (such as IL-12, IL-2, IL-11, IL-15, IL-18).

Fibroblasts encompassed herein may be derived from any tissue, including tissue selected from the group consisting of placenta, cord blood, mobilized peripheral blood, omentum, hair follicle, skin, bone marrow, adipose tissue, Wharton’s Jelly, and a combination thereof. In some embodiments, fibroblasts are derived from dermis, including dermis of any individual encompassed herein. In certain embodiments, fibroblasts are pretreated, such as to enhance immune modulatory activity, which may comprise an activity to suppress antigen presenting cell activity and/or activity to suppress an antigen presenting cell’s ability to stimulate a T cell response. The pretreatment may comprise exposure to at least one toll like receptor (TLR) agonist, such as at a sufficient concentration and time to enhance immune modulator activity of the fibroblasts. Times of exposure of agonists to cells may be between 1 second to 2 weeks, and may be around 24-48, 24-36, or 36-48 hours in some cases. In specific examples, the exposure of time is about 1 second to 1, 2, 3, 4, 5, 6, 7, 8, 9, 10, 11, 12, 13, or 14 days and any range therebetween. The exposure of time may be about 0.25, 0.5, 1, 2, 3, 4, 5, 6, 7, 8, 9, 10, 11, 12, 13, 14, 15, 16, 17, 18, 19, 20, 21, 22, 23 or more hours to 1, 2, 3, 4, 5, 6, 7, 8, 9, 10, 11, 12, 13, or 14 days and any range therebetween. The concentration of agonist ranges from about 1 picogram per ml to 1 mg/ml and any range therebetween. The concentration may be about 1 pg/ml to 0.25, 0.5, 0.75, or 1 mg/ml or about 1 (or 5, 10, 25, 50, 75, 100, 250, 500, 750, or more) pg/ml to 5, 10, 20, 25, 50, 75, 100, 150, 175, 200, 250, 500, or 750 or more µg/ml and any range therebetween. Particular concentrations depend on status of fibroblasts, for example, cycling fibroblasts may require higher concentration then senescent fibroblasts. This may be identified by one of skill in the art without undue experimentation and with reference to the prior art.

The TLR may be TLR-1, TLR-2, TLR-3, TLR-4, TLR-5, TLR-6, TLR-7, TLR-8, and/or TLR-9. The TLR-1 agonist may comprise Pam3CSK4. The TLR-2 agonist may comprise HKLM. The TLR-3 agonist may comprise Poly:IC. The TLR-4 agonist may be selected from the group consisting of LPS, buprenorphine, carbamazepine, fentanyl, levorphanol, methadone, cocaine, morphine, oxcarbazepine, oxycodone, pethidine, glucuronoxylomannan from Cryptococcus, morphine-3-glucuronide, lipoteichoic acid, β-defensin 2, small molecular weight hyaluronic acid, fibronectin EDA, snapin, tenascin C, and a combination thereof. The TLR-5 agonist may comprise flagellin. The TLR-6 agonist may comprise FSL-1. The TLR-7 agonist may comprise imiquimod. The TLR-8 agonist may comprise ssRNA40/LyoVec. The TLR-9 agonist may comprise CpG oligonucleotide, ODN2006, agatolimod, or a combination thereof.

Certain embodiments of the present disclosure concern administering fibroblasts to an individual, including an individual having, or at risk of having, an autoimmune disease. In some embodiments, fibroblasts are administered together with mesenchymal stem cells (MSCs), such as in a manner to allow MSCs to enhance immune modulatory effects of the fibroblasts. The immune modulatory effects may include suppression of: maturation of antigen presenting cells; NF-kappa b activity in antigen presenting cells; and/or production of IL-2, IL-12, IL-15, IL-18, or a combination thereof in antigen presenting cells. In some embodiments, T regulatory cell production is concurrently (such as at the time of administration fibroblasts or within a short amount of time (e.g., second or minutes) after administration of fibroblasts) increased with the administration of fibroblasts to an individual, which may enhance tolerogenic processes and/or reprogramming of antigen presenting cells towards a tolerogenic phenotype. The T regulatory cell production increase may be accomplished by administration of low dose IL-2 to an individual, such as at a dosage between 50,000 to 5,000,000 or between 500,000 to 5,000,000 or between 700,000 to 2,000,000 or between 1,000,000 to 2,000,000 IU per day. The IL-2 dosage may be 50,000, 500,000, 700,000, 1,000,000, 1,500,00, 2,000,000, or 5,000,000 IU per day or any range derivable therein.

Certain embodiments concern the induction of a tolerogenic loop in an individual. The embodiment of a tolerogenic loop comprises an immunological state in which T regulatory cells program dendritic cells to maintain an immature state, and the immature dendritic cells instruct the generation of new T regulatory cells. For the purpose of the disclosure, in one embodiment, induction of a tolerogenic loop comprises the steps of: a) administering a cell population comprising a therapeutic amount of fibroblasts (including, for example, any fibroblast encompassed herein) that possesses tolerogenic properties; b) augmenting ability of said tolerance-promoting fibroblast (after administration of the fibroblasts) to enhance generation of tolerogenic antigen presenting cells; and c) allowing for generation of T regulatory cells, wherein said immature tolerogenic antigen presenting cells allow for the generation of T regulatory cells. The individual may have, or be at risk of having, an autoimmune disease or transplant rejection. The fibroblasts may be pretreated with at least one activator of a TLR, including any agonist of TLR-1, TLR-2, TLR-3, TLR-4, TLR-5, TLR-6, TLR-7, TLR-8, and/or TLR-9 such as those disclosed herein, which may endow the fibroblast with tolerogenic properties. The pretreatment with the activator(s) of a TLR may result in the upregulation of CD200 on the fibroblast, which may result in sufficient expression of CD200 to block maturation of dendritic cells, including myeloid dendritic cells. Blocking the maturation of dendritic cells may result in the dendritic cells possessing low or absent levels of CD40, CD80, CD86, and/or IL-12. The pretreatment with the activator(s) of a TLR may result in the upregulation of HLA-G, IL-35, and/or IL-10 in the fibroblast.

In some embodiments, the fibroblasts, including the pretreated fibroblasts, are administered with mesenchymal stem cells, which may express CD73, CD90, and/or CD105 and may not express CD14, CD34, and/or HLA-DR. The MSCs may be derived from any tissue, including tissue selected from the group consisting of umbilical cord blood, Wharton’s Jelly, bone marrow, adipose tissue, menstrual blood, endometrial tissue, peripheral blood, deciduous teeth, mobilized peripheral blood, placenta, and a combination thereof. In some embodiments, the MSCs are used as a source of exosomes. The exosomes derived from MSCs may be added to the fibroblasts for endowment of tolerogenic properties to the fibroblasts.

Certain embodiments of the present disclosure concern methods for inducing tolerance in an individual. In some embodiments, a population of fibroblasts is obtained and treated under conditions to endow tolerance promoting properties. The treated fibroblasts are subsequently administered to an individual, including any individual encompassed herein.

The fibroblasts encompassed herein may proliferate at a rate of one double every 18-36 hours or 24-30 hours or any range derivable therein. The fibroblasts may produce 1-100 ng of IL-10 when cultured with dendritic cells at a concentration of 1 million fibroblasts and 1 million dendritic cells in a volume of 2 ml DMEM media supplemented with fetal calf serum. The fibroblasts may be cultured in a media allowing for proliferation of said fibroblasts, while augmenting immune modulatory activity of said fibroblasts. The media may contain n-acetylcysteine at a concentration between 0.01 to 100 µg/mL. The media may contain oxytocin at a concentration between 0.01 to 100 IU/mL. The media may contain IFNγ at a concentration between 0.001 to 100 IU/mL.

In some embodiments, an autoantigen is administered with fibroblasts to an individual. The autoantigen may be comprised of any protein, peptide, or altered peptide ligand, including those derived from proteins that are involved in autoimmune diseases. In some embodiments, one or more autoantigens is expressed, such as in a constitutive or inducible manner, in the fibroblast. Fibroblasts expressing the autoantigen(s) may be modified to express one or more tolerance promoting molecules, such as a molecules that induce death of autoreactive T cells including FAS ligand, TNF-alpha, TNF-beta, TRAIL, granzyme, perforin, or a combination thereof, or molecules that induce the generation of T regulatory cells including IL-10, HLA-G, TGF-beta, IL-35, or a combination thereof.

The fibroblasts and fibroblast populations encompassed herein may comprise autologous, allogeneic, and/or xenogeneic fibroblasts relative to any individual encompassed herein, including an individual having, or at risk of having, an autoimmune disease or transplant rejection and including an individual receiving the administration of fibroblasts.

### II. Definitions

Unless defined otherwise, all technical and scientific terms used herein have the same meaning as commonly understood by one of ordinary skill in the art to which the invention pertains. The articles “a” and “an” are used herein to refer to one or to more than one (i.e., to at least one) of the grammatical object of the article. By way of example, “an element” means one element or more than one element.

“About” or “approximately” as used herein when referring to a measurable value such as an amount, a temporal duration, and the like, is meant to encompass variations of +/-20% or in some instances +/- 10%, or in some instances +/- 5%, or in some instances +/- 1%, or in some instances +/- 0.1% from the specified value, as such variations are appropriate to perform the disclosed methods.

“Activation,” as used herein typically when referring to cells, such as T cells, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division. Furthermore “activated DC” implies a DC that possess ability to provide Signal I (MHC and antigen) as well as Signal II (costimulatory signals) to T cells, thus allowing for the activation of T cells, for example. In the case of conventional T cells, activation of CD4 cells implies augmentation of cytokine production. In the case of CD8 T cells, activation implies enhancement of cytotoxic activity. In the case of T regulatory cells, activation implies augmentation of suppressive activity to other immune cells.

“Administering” as used herein, refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, and intrasternal injection, infusion, and/or in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “antibody,” as used herein, refers to an immunoglobulin molecule which specifically binds with an antigen. Antibodies can be intact immunoglobulins derived from natural sources or from recombinant sources. Antibodies can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies. The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments. An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. κ and λ light chains refer to the two major antibody light chain isotypes.

The term “synthetic antibody” as used herein, refers to an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “antigen presenting cell” as used herein refers to any cell capable of presenting at least one antigen, including any antigen encompassed herein, in order to provoke an immune response. The antigen presenting cell may be, as non-limiting examples, a dendritic cell, macrophage, B cell, or innate lymphoid cell. The term “antigen presenting cell” or “antigen presenting cells” as used herein may also refer to, as non-limiting examples, a plurality of different cell types, including a plurality of dendritic cells, macrophages, B cells, innate lymphoid cells, endothelial cells, or a combination thereof. Antigen presenting cells may or may not stimulate pathogenic immune responses. Antigen presenting cells may or may not be tolerogenic and/or stimulate tolerogenic cells.

The term “auto-antigen” as used herein refers to any self-antigen which is mistakenly recognized by the immune system of an individual as being foreign. Auto-antigens comprise, but are not limited to, cellular proteins, phosphoproteins, cellular surface proteins, cellular lipids, nucleic acids, glycoproteins, including cell surface receptors, which may be endogenous to the individual. Auto-antigens may be any peptide derived from a protein endogenous to the individual.

As used herein, the term “autologous” is meant to refer to any material derived from the same individual to which it is later to be re-introduced into the individual.

The term “fibroblast” defines, intra alia, cells from various tissues, selected for specific properties associated with regenerative activity, wherein the regenerative properties include at least the ability to differentiate into other tissues as well as produce growth factors. Tissues useful for the practice of the disclosure are generally tissues associated with regenerative activity. Such tissues include placenta, endometrial cells, Wharton’s jelly, bone marrow, and adipose tissue for example. In some embodiments, cells are selected for expression of the markers CD117, CD105, and for expression of the rhodamine 123 efflux activity. In some embodiments, fibroblasts are selected for expression of markers selected from the group consisting of the additional markers Oct-4, CD-34, KLF-4, Nanog, Sox-2, Rex-1, GDF-3, Stella, and that they possess enhanced expression of GDF-11.

“Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineages. Other cells possessing mesenchymal-like properties are included within the definition of “mesenchymal stem cell”, with the condition that said cells possess at least one of the following: a) regenerative activity; b) production of growth factors; and c) ability to induce a healing response, either directly, or through elicitation of endogenous host repair mechanisms. As used herein, “mesenchymal stromal cell” or ore mesenchymal stem cell can be used interchangeably.

The term “T regulatory cells” or “Tregs” define a cell population that plays a critical role in the maintenance of peripheral self-tolerance. Naturally occurring CD4⁺ CD25^{hi} Tregs are produced in the thymus and express FoxP3, a transcriptional factor required for establishment and maintenance of Treg lineage identity and suppressor function. Tregs can accumulate at a disease site, where they suppress the effector function of disease specific T cells. When this occurs it can result in an increase in disease despite the presence of appropriate antigens or T cells activated to attack those antigens. Increased densities of tumor-infiltrating FoxP3⁺ Tregs have been associated with poor prognosis in various solid tumors, including pancreatic, ovarian, and hepatocellular carcinoma. Depletion of Tregs results in enhanced antitumor immunity and tumor rejection in murine models but may also result in the development of autoimmune diseases. For certain embodiments of the disclosure, the utilization of fibroblasts to modify antigen presenting cell activities is performed in order to, intra alia, treat autoimmunity or transplant rejection through stimulation of Treg activity. The Treg may directly inhibit other T cells from helping, or may directly suppress cytotoxic T cells. Additionally, Treg generated by dendritic cells made immature using fibroblasts and/or mesenchymal stem cells may in turn inhibit generation of immune activatory antigen presenting cells, leading to formation of a self-feeding tolerogenic loop.

### III. Pathological Immune Response

Certain embodiments of the present disclosure concern methods for inhibiting a pathological immune response. The pathological immune response may be any immune response in which immune cells aberrantly recognize an auto-antigen as a foreign antigen and activate a pathologic and/or cytotoxic immune response, including a pathologic and/or cytotoxic immune response against cells that express the auto-antigen. The pathological immune response may comprise at least one autoimmune reaction, autoimmune disease, graft rejection, graft versus host disease, host versus graft disease, or a combination thereof. In some embodiments, the pathological immune response comprises an in vivo response. In some embodiments, the pathological immune response comprises an in vitro response.

Certain embodiments concern methods for inducing tolerance in an individual. In some embodiments the individual has, or is at risk of having, a pathological immune response, including any pathological immune response encompassed herein.

Certain embodiments concern methods for treating an individual having, or at risk of having, a pathological immune response, including any pathological immune response encompassed herein.

Certain embodiments concern methods for administering therapeutic compositions, which may or may not comprise a therapeutically effective amount of one or more cell types, including fibroblasts (which may or may not be modified and/or pretreated) and/or mesenchymal stem cells (which may or may not be modified and/or pretreated), to an individual (or cells from an individual) having, or at risk of having, a pathological immune response, including any pathological immune response encompassed herein. In some embodiments, the therapeutic compositions comprises exosomes, including any exosomes encompassed herein. The therapeutic composition may modify and/or inhibit antigen presenting cells in or from the individual.

Embodiments of the present disclosure include methods of treating a pathological immune response in an individual. The pathological immune response may be of any kind and from any cause, but in specific embodiments it comprises at least one autoimmune reaction, autoimmune disease, graft rejection, graft versus host disease, host versus graft disease, or a combination thereof. In such cases, an individual that has a pathological immune response or is at risk for having a pathological immune response is provided an effective amount of fibroblasts and/or MSCs as described herein.

### IV. Fibroblasts

Certain embodiments of the present disclosure concern the previously unknown property of fibroblasts to alter the ability of antigen presenting cells from an immune stimulatory role to an immune inhibitory and/or tolerogenic role. Certain embodiments concern methods for the treatment of conditions in which suppression of an immune response is desired, for example an autoimmune or alloimmune (such as a graft rejection) response. By administration of manipulated and/or non-manipulated fibroblasts directly into a host, some embodiments provide means of suppressing immunity by promoting the ability of antigen presenting cells to stimulate tolerogenic immunological mechanisms including induction of T cell anergy, stimulation of T regulatory cell production, and blockade of costimulatory molecule expression. In some embodiments, combinations of fibroblasts and mesenchymal stem cells (MSC) are utilized in order for induction of a state of antigen-nonspecific as well as antigen-specific immune modulation.

In some embodiments, unmodified fibroblasts and/or fibroblasts that have been modified are utilized for induction of a tolerogenic program in vitro and/or in vivo, for generation of antigen presenting cells that possess tolerogenic properties. In some embodiments, fibroblasts are utilized to generate a population in vitro or in vivo of antigen presenting cells, including dendritic cells (DCs), which reprogram the immune system towards tolerance and away from activation. DCs are classically known to act as master sentinels of the immune system, being the only cell capable of activating naive T cells (1-5). This unique ability is in part endowed by expression of a unique sent of membrane bound and soluble molecules that act as costimulatory signals (6,7). Additionally, DCs interact with other antigen presenting cells in the process of initiating, refining, and fine-tuning T cell responses (8-10). Interestingly, DCs possess not only constitutive expression of such molecules, but also have inducible expression depending on the needs of the body (11). In some embodiments, fibroblasts and/or fibroblasts combined with fibroblast derived exosomes, and/or fibroblasts combined with mesenchymal stem cells, and/or fibroblasts combined with mesenchymal stem cell derived exosomes are utilized to allow for generation of tolerogenic antigen presenting cells, in particular, tolerogenic dendritic cells. In some embodiments, fibroblasts are generated to possess tolerogenic, or antigen presenting cell modulatory properties by incubation of fibroblasts with toll like receptor (TLR) agonists.

There are multiple types of DCs that may be modified by fibroblasts. In some embodiments, fibroblasts endow tolerogenic activity on various DCs from the 4 main categories of DCs, which are plasmacytoid DCs, cDC1, cDC2, monocyte derived DCs, which are associated with inflammation. It is believed that human plasmacytoid DC act as one of the first lines of defenses against viruses, in part, by producing large amounts of interferon alpha and interferon beta (12-17). These cells have been described under of variety of names including, plasmacytoid T cells (18-24), plasmacytoid monocytes (25), natural IFN-a/β-producing cells (26-28). Some evidence suggests that plasmacytoid DCs are capable of being generated both from myeloid and lymphoid progenitors (29,30).

The production of interferons by plasmacytoid DC is controlled by other cytokines in the periphery of the cells, for example, monocytes produced IFN-I in response to Sendai virus (SV) infection, and PDC responded to both SV and herpes simplex virus (HSV). All cytokines tested failed to induce production of IFN-I in the absence of infection. However, among 18 relevant cytokines, incubation of PDC with interleukin-4 (IL-4), IL-15, and IL-7 alone or in combination with IL-3 before infection, enhanced IFN-I secretion. At variance, IL-12 alone or in synergy with granulocyte-macrophage colony-stimulating factor (GM-CSF) was active on SV-infected but not on HSV-infected monocytes. Tumor necrosis factor-alpha (TNF-alpha) and IL-4 inhibited IFN-I production by PDC and monocytes, respectively, and IL-10 strongly inhibited IFN-I production in both cell lineages. The response of PDC to IL-7 and IL-15, which also activate natural killer (NK) cell maturation, further emphasizes the cooperation between these two cell subsets in the control of innate immunity (31).

The relevance of plasmacytoid DC in host immunity to viruses is evident by experiments in which Dengue, an acute flavivirus disease, is used as a model to study DC responses to a self-limited human viral infection. Investigators analyzed circulating DC subsets in a prospective study of children with dengue across a broad range of illness severities: healthy controls; mild, non-dengue, presumed viral infections; moderately ill dengue fever; and, the most severe form of illness, dengue hemorrhagic fever. We also examined PDC responses in monkeys with asymptomatic dengue viremia and to dengue virus exposure in vitro. The absolute number and frequency of circulating pre-mDCs early in acute viral illness decreased as illness severity increased. Depressed pre-mDC blood levels appeared to be part of the typical innate immune response to acute viral infection. The frequency of circulating PDCs trended upward and the absolute number of circulating PDCs remained stable early in moderately ill children with dengue fever, mild other, non-dengue, febrile illness, and monkeys with asymptomatic dengue viremia. However, there was an early decrease in circulating PDC levels in children who subsequently developed dengue hemorrhagic fever. A blunted blood PDC response to dengue virus infection was associated with higher viremia levels, and was part of an altered innate immune response and pathogenetic cascade leading to severe disease (32).

In some embodiments, fibroblasts are administered in a manner to augment the tolerogenic properties of endothelial cells. The presentation of antigens by endothelial cells has been previously described (33,34). In some embodiments of the invention, fibroblasts are administered together with one or more agents capable of mobilizing DC and/or DC precursors in order to endow a tolerogenic state in said mobilized DC. The ability to selectively mobilize one type of DC versus another type was demonstrated by Pulendran et al who reported that administration of either Flt3-ligand (FL) or G-CSF to healthy human volunteers dramatically increases distinct DC subsets, or DC precursors, in the blood. FL increases both the CD11c+ DC subset (48-fold) and the CD11c- IL-3R+ DC precursors (13-fold). In contrast, G-CSF only increases the CD11c- precursors (>7-fold). Freshly sorted CD11c+ but not CD11c- cells stimulate CD4+ T cells in an allogeneic MLR, whereas only the CD11c- cells can be induced to secrete high levels of IFN-alpha, in response to influenza virus. CD1lc+ and CD11c- cells can mature in vitro with GM-CSF + TNF-alpha or with IL-3 + CD40 ligand, respectively. These two subsets up-regulate MHC class II costimulatory molecules as well as the DC maturation marker DC-lysosome-associated membrane protein, and they stimulate naive, allogeneic CD4+ T cells efficiently. These two DC subsets elicit distinct cytokine profiles in CD4+ T cells, with the CD11c- subset inducing higher levels of the Th2 cytokine IL-10. The differential mobilization of distinct DC subsets or DC precursors by in vivo administration of FL and G-CSF offers a novel strategy to manipulate immune responses in humans (35).

In some embodiments, inhibition of DC function through blocking generation of mature DCs is accomplished by combination of standard DC maturation inhibiting protocols together with administration of fibroblasts. For the practice of some embodiments encompassed herein, DCs may be generated in one or several ways. Means of generating DC are known in the art and described in the following publications which are incorporated by reference: Generated from mobilized CD34 cells (36). In one embodiment, fibroblasts are utilized to endow plasmacytoid DCs with the ability to generate T regulatory cells, which are specific to autoantigen to which inhibition of immunity is desired. In one embodiment, conditions are generated similar to the conditions observed in a tumor microenvironment in order to allow for enhanced generation of tolerogenic cells. These conditions include acidity, hypoxia, and the presence of immune suppressive membrane bound molecules such as PD-L1 and CTLA-4, as well as soluble immune suppressive molecules such as IL-10 and TGF-beta. In one paper it was shown that ascites macrophage-derived dendritic cells induced tumor-associated antigen-specific CD8+ T cells with effector functions. Strikingly, tumor ascites plasmacytoid dendritic cells induced interleukin-10+ CCR7+ CD45RO+ CD8+ regulatory T cells. Four characteristics have been identified in tumor plasmacytoid dendritic cell-induced CD8+ regulatory T cells: (a) induction of CD8+ regulatory T cells is independent of CD4+ CD25+ T cells; (b) CD8+ regulatory T cells significantly suppress myeloid dendritic cell-mediated tumor-associated antigen-specific T cell effector functions through interleukin-10; (c) repetitive myeloid dendritic cell stimulation can recover CD8+ regulatory T cell-mediated poor T cell proliferation, but not T cell effector function; (d) CD8+ regulatory T cells express functional CCR7, and efficiently migrate with lymphoid homing chemokine MIP-3beta. Primary suppressive CCR7+ CD45RO+ CD8+ T cells are found in the tumor environment of patients with ovarian cancers. Thus, tumor-associated plasmacytoid dendritic cells contribute to the tumor environmental immunosuppressive network (37). Accordingly, in some embodiments, fibroblasts are treated with compounds associated with tumor immune suppression in order to enhance and/or endow fibroblasts tolerogenic properties similar to those found in tumor associated fibroblasts. Molecules or conditions useful for culturing fibroblasts for certain embodiments of the disclosure include PGE-2 (38-93), soluble HLA-G (94-112), IL-10, TGF-beta, acidic conditions, and hypoxic conditions. In some embodiments, the concentrations of immune modulatory agents are based on factors associated with endowment of immune modulatory properties, for example, 1 µM of PGE-2 is reported to stimulate stabilization of HIF-1 alpha (113,114). In some embodiments, PGE-2 concentrations that stimulate stabilization of HIF-1 alpha are desired for the practice of the disclosure.

Fibroblasts encompassed in certain embodiments herein may be generated by outgrowth from a biopsy of the recipient’s own skin (in the case of autologous preparations), or skin of healthy donors (for allogeneic preparations), for example. In some embodiments fibroblasts are used from young donors. In certain embodiments, fibroblasts are transfected with genes to allow for enhanced growth and overcoming of the Hayflick limit. Subsequent to derivation of cells expansion in culture using standard cell culture techniques. Skin tissue (dermis and epidermis layers) may be biopsied from a subject’s post-auricular area. In one embodiment, the starting material is composed of three 3-mm punch (or 1-2 mm smaller or 1-2 mm larger) skin biopsies collected using standard aseptic practices. The biopsies may be collected by the treating physician, placed into a vial containing sterile phosphate buffered saline (PBS). The biopsies may be shipped in a 2-8° C. refrigerated shipper back to the manufacturing facility. In one embodiment, after arrival at the manufacturing facility, the biopsy is inspected and, upon acceptance, transferred directly to the manufacturing area. Upon initiation of the process, the biopsy tissue may then be washed prior to enzymatic digestion. After washing, a Liberase Digestive Enzyme Solution may be added without mincing, and the biopsy tissue is incubated at 37.0 +/- 2.0° C. for approximately one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany).

After digestion, Initiation Growth Media (IMDM, GA, 10% Fetal Bovine Serum (FBS)) may be added to neutralize the enzyme, cells may then be pelleted by centrifugation and resuspended in approximately 5 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells may be incubated at 37.0 +/- 2.0° C. with 5.0 +/- 1.0% CO₂ and fed with fresh Complete Growth Media every three to five days. All feeds in the process may be performed by removing half of the Complete Growth Media and replacing the same volume with fresh media. Alternatively, full feeds can be performed. Cells should not remain in the T-175 flask greater than 30 days prior to passaging. Confluence may be monitored throughout the process to ensure adequate seeding densities during culture splitting.

When cell confluence is greater than or equal to 40% in the T-175 flask, the cells may be passaged by removing the spent media, washing the cells, and treating with Trypsin-EDTA to release adherent cells in the flask into the solution. Cells may then be trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology may be evaluated at each passage and prior to harvest to monitor the culture purity throughout the culture purity throughout the process. Morphology may be evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures may also be evaluated. Keratinocytes may appear round and irregularly shaped and, at higher confluence, they may appear organized in a cobblestone formation. At lower confluence, keratinocytes may be observable in small colonies. Cells may be incubated at 37.0 +/- 2.0° C. with 5.0 +/- 1.0% CO₂ and passaged every three to five days in the T-500 flask and every five to seven days in the ten layer cell stack (10 CS). Cells should not remain in the T-500 flask for more than 10 days prior to passaging.

Quality Control (QC) release testing for safety of the Bulk Drug Substance includes sterility and endotoxin testing. When cell confluence in the T-500 flask is >95%, cells may be passaged to a 10 CS culture vessel. Alternately, two Five Layer Cell Stacks (5 CS) or a 10 Layer Cell Factory (10 CF) can be used in place of the 10 CS. Passage to the 10 CS may be performed by removing the spent media, washing the cells, and treating with Trypsin-EDTA to release adherent cells in the flask into the solution. Cells may then be transferred to the 10 CS. Additional Complete Growth Media may be added to neutralize the trypsin and the cells from the T-500 flask are pipetted into a 2 L bottle containing fresh Complete Growth Media. The contents of the 2 L bottle may be transferred into the 10 CS and seeded across all layers. Cells may then be incubated at 37.0 +/- 2.0° C. with 5.0 +/- 1.0% CO₂ and fed with fresh Complete Growth Media every five to seven days. Cells should not remain in the 10 CS for more than 20 days prior to passaging.

In one embodiment, the passaged dermal fibroblasts are rendered substantially free of immunogenic proteins present in the culture medium by incubating the expanded fibroblasts for a period of time in protein free medium. When cell confluence in the 10 CS is 95% or more, cells may be harvested. Harvesting may be performed by removing the spent media, washing the cells, treating with Trypsin-EDTA to release adherent cells into the solution, and adding additional Complete Growth Media to neutralize the trypsin. Cells may be collected by centrifugation, resuspended, and in-process QC testing performed to determine total viable cell count and cell viability.

In some embodiments, when large numbers of cells are required after receiving cell count results from the primary 10 CS harvest, an additional passage into multiple cell stacks (up to four 10 CS) is performed. For additional passaging, cells from the primary harvest may be added to a 2 L media bottle containing fresh Complete Growth Media. Resuspended cells may be added to multiple cell stacks and incubated at 37.0 +/- 2.0° C. with 5.0 +/- 1.0% CO₂. The cell stacks may be fed and harvested as described above, except cell confluence must be 80% or higher prior to cell harvest. The harvest procedure may be the same as described for the primary harvest above. A mycoplasma sample from cells and spent media may be collected, and cell count and viability performed as described for the primary harvest above. The method may decrease or eliminate immunogenic proteins by avoiding their introduction from animal-sourced reagents. To reduce process residuals, cells may be cryopreserved in protein-free freeze media, then thawed and washed prior to prepping the final injection to further reduce remaining residuals. If additional Drug Substance is needed after the harvest and cryopreservation of cells from additional passaging is complete, aliquots of frozen Drug Substance--Cryovial are thawed and used to seed 5 CS or 10 CS culture vessels. Alternatively, a four layer cell factory (4 CF), two 4 CF, or two 5 CS can be used in place of a 5 CS or 10 CS. A frozen cryovial(s) of cells is thawed, washed, added to a 2 L media bottle containing fresh Complete Growth Media and cultured, harvested and cryopreserved as described above. The cell suspension is added Cell confluence must be 80% or more prior to cell harvest.

At the completion of culture expansion, the cells are harvested and washed, then formulated to contain 1.0-2.7 x 10⁷ cells/mL, with a target of 2.2 x 10⁷ cells/mL. Alternatively, the target can be adjusted within the formulation range to accommodate different indication doses. The drug substance consists of a population of viable, autologous human fibroblast cells suspended in a cryopreservation medium consisting of Iscove’s Modified Dulbecco’s Medium (IMDM) and Profreeze-CDM™ (Lonza, Walkerville, Md.) plus 7.5% dimethyl sulfoxide (DMSO). Alternatively, a lower DMSO concentration may be used in place of 7.5% or CryoStor™ CS5 or CryoStor™ CS10 (BioLife Solutions, Bothell, Wash.) may be used in place of IMDM/Profreeze/DMSO. In addition to cell count and viability, purity/identity of the Drug Substance may be performed and must confirm the suspension contains 98% or more fibroblasts. The usual cell contaminants include keratinocytes. The purity/identify assay employs fluorescent-tagged antibodies against CD90 and CD 104 (cell surface markers for fibroblast and keratinocyte cells, respectively) to quantify the percent purity of a fibroblast cell population. CD90 (Thy-1) is a 35 kDa cell-surface glycoprotein. Antibodies against CD90 protein have been shown to exhibit high specificity to human fibroblast cells. CD104, integrin β4 chain, is a 205 kDa transmembrane glycoprotein which associates with integrin α6 chain (CD49f) to form the α6/(β4 complex. This complex has been shown to act as a molecular marker for keratinocyte cells (Adams and Watt 1991).

Antibodies to CD 104 protein bind to approximately 100% of human keratinocyte cells. Cell count and viability is determined by incubating the samples with Viacount Dye Reagent and analyzing samples using the Guava PCA system. The reagent is composed of two dyes, a membrane-permeable dye which stains all nucleated cells, and a membrane-impermeable dye which stains only damaged or dying cells. The use of this dye combination enables the Guava PCA system to estimate the total number of cells present in the sample, and to determine which cells are viable, apoptotic, or dead. The method was custom developed specifically for use in determining purity/identity of autologous cultured fibroblasts. Alternatively, cells can be passaged from either the T-175 flask (or alternatives) or the T-500 flask (or alternatives) into a spinner flask containing microcarriers as the cell growth surface. Microcarriers are small bead-like structures that are used as a growth surface for anchorage dependent cells in suspension culture. They are designed to produce large cell yields in small volumes. In this apparatus, a volume of Complete Growth Media ranging from 50 mL-300 mL may be added to a 500 mL, 1 L or 2 L sterile disposable spinner flask. Sterile microcarriers may be added to the spinner flask. The culture may be allowed to remain static or may be placed on a stir plate at a low RPM (such as at approximately 15-30 RRM) for a short period of time (1-24 hours) in a 37.0 +/- 2.0° C. with 5.0 +/- 1.0% CO₂ incubator to allow for adherence of cells to the carriers.

After the attachment period, the speed of the spin plate may be increased (such as to approximately 30-120 RPM). Cells may be fed with fresh Complete Growth Media every one to five days, or when media appears spent by color change. Cells may be collected at regular intervals by sampling the microcarriers, isolating the cells and performing cell count and viability analysis. The concentration of cells per carrier may be used to determine when to scale-up the culture. When enough cells are produced, cells may be washed with PBS and harvested from the microcarriers using trypsin-EDTA and seeded back into the spinner flask in a larger amount of microcarriers and higher volume of Complete Growth Media (300 mL-2 L). Alternatively, additional microcarriers and Complete Growth Media can be added directly to the spinner flask containing the existing microcarrier culture, allowing for direct bead-to-bead transfer of cells without the use of trypsinization and reseeding. Alternatively, if enough cells are produced from the initial T-175 or T-500 flask, the cells can be directly seeded into the scale-up amount of microcarriers. After the attachment period, the speed of the spin plate may be increased (such as to approximately 30-120 RPM). Cells are fed with fresh Complete Growth Media every one to five days, or when media appears spent by color change. When the concentration reaches the desired cell count for the intended indication, the cells are washed with PBS and harvested using trypsin-EDTA. Microcarriers used within the disposable spinner flask may be made from poly blend such as BioNOC II.RTM. (Cesco Bioengineering, distributed by Bellco Biotechnology, Vineland, N.J.) and FibraCel.RTM. (New Brunswick Scientific, Edison, N.J.), gelatin, such as Cultispher-G (Percell Biolytica, Astrop, Sweden), cellulose, such as Cytopore™ (GE Healthcare, Piscataway, N.J.) or coated/uncoated polystyrene, such as 2D MicroHex™ (Nunc, Weisbaden, Germany), Cytodex® (GE Healthcare, Piscataway, N.J.) or Hy-Q Sphere™ (Thermo Scientific Hyclone, Logan, Utah).

In one embodiment, fibroblasts are preactivated by contact with a growth factor containing mixture, said mixture, or composition comprises growth factors selected from the group consisting of transforming growth factors (TGF), fibroblast growth factors (FGF), platelet-derived growth factors (PDGF), epidermal growth factors (EGF), vascular endothelial growth factors (VEGF), insulin-like growth factors (IGF), platelet-derived endothelial growth factors (PDEGF), platelet-derived angiogenesis factors (PDAF), platelet factors 4 (PF-4), hepatocyte growth factors (HGF) and mixtures thereof. In some embodiments, the growth factors are transforming growth factors (TGF), platelet-derived growth factors (PDGF) fibroblast growth factors (FGF) or mixtures thereof. In some embodiments, the growth factors are selected from the group consisting of transforming growth factors β (TGF-β), platelet-derived growth factors BB (PDGF-BB), basic fibroblast growth factors (bFGF) and mixtures thereof. In certain embodiments, said growth factor containing compositions are injected simultaneously with, or subsequent to, injection of fibroblasts. Said fibroblasts may be autologous, allogeneic, or xenogeneic to an individual.

### V. Mesenchymal Stem Cells

Mesenchymal stem (or stromal) cells (MSCs) can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may include cells that are isolated from tissues using cell surface markers selected from the group consisting of NGF-R, PDGF-R, EGF-R, IGF-R, CD29, CD49a, CD56, CD63, CD73, CD105, CD106, CD140b, CD146, CD271, MSCA-1, SSEA4, STRO-1, STRO-3 and combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn®, Stemedyne®-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

MSCs may be expanded and utilized by administration themselves, or may be cultured in a growth media in order to obtain conditioned media, the term Growth Medium generally refers to a medium sufficient for the culturing of umbilicus-derived (as one example) cells.

Mesenchymal stem cells (“MSC”) were originally derived from the embryonal mesoderm and subsequently have been isolated from adult bone marrow and other adult tissues. They can be differentiated to form muscle, bone, cartilage, fat, marrow stroma, and tendon. Mesoderm also differentiates into visceral mesoderm which can give rise to cardiac muscle, smooth muscle, or blood islands consisting of endothelium and hematopoietic progenitor cells. The differentiation potential of the mesenchymal stem cells that have been described thus far is limited to cells of mesenchymal origin, including the best characterized mesenchymal stem cell (See Pittenger, et al. Science (1999) 284: 143-147 and U.S. Pat. No. 5,827,740 (SH2⁺ SH4⁺ CD29⁺ CD44⁺ CD71⁺ CD90⁺ CD106⁺ CD120a⁺ CD124⁺ CD14⁻ CD34⁻ CD45⁻)). Certain embodiments of the disclosure concern the use of various mesenchymal stem cells.

In one embodiment, MSC donor lots are generated from umbilical cord tissue. Means of generating umbilical cord tissue MSC have been previously published and are incorporated by reference (115-121). The term “umbilical tissue derived cells” or “UTC” refers, for example, to cells as described in U.S. Pat. No. 7,510,873, U.S. Pat. No. 7,413,734, U.S. Pat. No. 7,524,489, and U.S. Pat. No. 7,560,276. The UTC can be of any mammalian origin e.g. human, rat, primate, porcine and the like. In one embodiment, the UTC are derived from human umbilicus. Umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle).

In addition, these isolated human umbilicus-derived cells express a gene for each of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes. Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes. Tissue can be obtained from any completed pregnancy, term or less than term, whether delivered vaginally, or through other routes, for example surgical Cesarean section. Obtaining tissue from tissue banks is also considered within the scope of the present disclosure. The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment. Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In some embodiments, in order to determine the quality of MSC cultures, flow cytometry is performed on all cultures for surface expression of SH-2, SH-3, SH-4 MSC markers and lack of contaminating CD14- and CD-45 positive cells. Cells may be detached with 0.05% trypsin-EDTA, washed with DPBS + approximately 2% bovine albumin, fixed in approximately 1% paraformaldehyde, blocked in approximately 10% serum, incubated separately with primary SH-2, SH-3 and SH-4 antibodies followed by PE-conjugated anti-mouse IgG(H+L) antibody. Confluent MSCs in 175 cm² flasks may be washed with Tyrode’s salt solution, incubated with medium 199 (M199) for approximately 60 min, and detached with 0.05% trypsin-EDTA (Gibco).

### VI. Exosomes

Certain embodiments concern modulating antigen presenting cells, including dendritic cells, to promote tolerogenesis in an individual. In some embodiments, exosomes derived from fibroblasts and/or MSCs that have been activated with toll like receptor agonists are utilized. For example, fibroblasts may be cultured with lipopolysaccharide, such as at a concentration of 1, 2, 3, 4, 5, 6, 7, 8, 9, 10, or any range derivable therein µg/mL. Exosomes resulting from the fibroblasts and/or MSCs may then be purified. The exosomes may possess enhanced ability to inhibited DC maturation, as well as to promote generation of T regulatory cells. The isolation of exosomes may be performed using means known in the art. In some embodiments, exosomes are purified by liquid chromatography. In some embodiments, exosomes are purified by sedimentation using ultracentrifugation. In some embodiments exosomes are isolated based on size filtration.

Exosomes, also referred to herein as “particles” may comprise vesicles or a flattened sphere limited by a lipid bilayer. The particles may comprise diameters of 40-100 nm. The particles may be formed by inward budding of the endosomal membrane. The particles may have a density of about 1.13-1.19 g/mL and may float on sucrose gradients. The particles may be enriched in cholesterol and sphingomyelin, and lipid raft markers such as GM1, GM3, flotillin and the src protein kinase Lyn. The particles may comprise one or more proteins present in fibroblasts or fibroblast conditioned medium (F-CM), such as a protein characteristic or specific to the fibroblasts or media conditioned by fibroblasts. They may comprise RNA, for example miRNA. The particles may possess one or more genes or gene products (such as EGF, IGF-1, HGF, FGF-1, FGF-2, FGF-5, PDGF, and angiopoietin) found in fibroblasts or medium which is conditioned by culture of fibroblasts. The particle may comprise molecules secreted by the fibroblast. Such a particle, and combinations of any of the molecules comprised therein, including in particular proteins or polypeptides, may be used to supplement the activity of, or in place of, the fibroblast or medium conditioned by the fibroblast for the purpose of for example treating or preventing a disease. Said particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular, the particle may comprise one or more tetraspanins. The particles may comprise mRNA and/or microRNA. The particle may be used for any of the therapeutic purposes that the fibroblast or media conditioned by fibroblasts may be put to use.

In some embodiments, fibroblast exosomes or particles may be produced by culturing said fibroblast in a medium to condition it. The fibroblast may be derived from human dermal tissue which possess markers selected from a group comprising of CD90, CD73 and CD105. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/mL FGF2. The concentration of PDGF AB may be about 5 ng/mL. The medium may comprise glutamine-penicillin-streptomycin or b-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. It may be concentrated by filtration through a membrane. The membrane may comprise a >1000 kDa membrame. The conditioned medium may be concentrated about 50 times or more.

The conditioned medium may be subject to liquid chromatography such as HPLC. The conditioned medium may be separated by size exclusion. Any size exclusion matrix such as Sepharose may be used. As an example, a TSK Guard column SWXL, 6x40 mm or a TSK gel G4000 SWXL, 7.8x300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 mL/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The rₕ of particles in this peak is about 45-55 nm. Such fractions comprise fibroblast particles such as exosomes.

### VII. Harvesting Cells and Growth Conditions

In some embodiments, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. In some embodiments, enzyme activites are selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease may be used in a sequence following the use of other enzymes as they may degrade the other enzymes being used. In certain embodiments, the temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Certain embodiments involve enzymatic treatment with, for example, collagenase and dispase, or collagenase, dispase, and hyaluronidase, and such methods are provided wherein in certain embodiments, a mixture of collagenase and the neutral protease dispase are used in the dissociating step. Certain embodiments employ digestion in the presence of at least one collagenase from Clostridium histolyticum, and either of the protease activities, dispase and thermolysin. Certain embodiments employ digestion with both collagenase and dispase enzyme activities. Also encompassed are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Certain enzyme treatments are 0.5, 1, 1.5, 2, or any range derivable therein hours long or longer. In some embodiments, the tissue is incubated at approximately 37° C. during the enzyme treatment of the dissociation step. Diluting the digest may also improve yields of cells as cells may be trapped within a viscous digest. While the use of enzyme may be performed, it is not required for isolation methods as provided herein. Methods based on mechanical separation alone may be successful in isolating the instant cells from the umbilicus as discussed above. The cells can be resuspended after the tissue is dissociated into any culture medium as discussed herein above.

Cells may be resuspended following a centrifugation step to separate out the cells from tissue or other debris. Resuspension may involve mechanical methods of resuspending, or simply the addition of culture medium to the cells. Providing the growth conditions allows for a wide range of options as to culture medium, supplements, atmospheric conditions, and relative humidity for the cells. A preferred temperature is 3° C., however the temperature may range from about 3° C. to 3° C. depending on the other culture conditions and desired use of the cells or culture.

In certain embodiment, cells can be processed on poly blend 2D microcarriers such as BioNOC II® and FibraCel® using an automatic bellow system, such as FibraStage™ (New Brunswick Scientific, Edison, N.J.) or BelloCell®. (Cesco Bioengineering, distributed by Bellco Biotechnology, Vineland, N.J.) in place of the spinner flask apparatus. Cells from the T-175 (or alternatives) or T-500 flask (or alternatives) are passaged into a bellow bottle containing microcarriers with the appropriate amount of Complete Growth Media, and placed into the system. The system may pump media over the microcarriers to feed cells, and draws away media to allow for oxygenation in a repeating fixed cycle. Cells may be monitored, fed, washed and harvested in the same sequence as described above. Alternatively, cells can be processed using automated systems. After digestion of the biopsy tissue or after the first passage is complete (T-175 flask or alternative), cells may be seeded into an automated device. One method is an Automated Cellular Expansion (ACE) system, which is a series of commercially available or custom fabricated components linked together to form a cell growth platform in which cells can be expanded without human intervention. Cells may be expanded in a cell tower, consisting of a stack of disks capable of supporting anchorage-dependent cell attachment. The system automatically circulates media and performs trypsinization for harvest upon completion of the cell expansion stage.

Alternatively, the ACE system can be a scaled down, single lot unit version comprised of a disposable component that consists of cell growth surface, delivery tubing, media and reagents, and a permanent base that houses mechanics and computer processing capabilities for heating/cooling, media transfer and execution of the automated programming cycle. Upon receipt, each sterile irradiated ACE disposable unit may be unwrapped from its packaging and loaded with media and reagents by hanging pre-filled bags and connecting the bags to the existing tubing via aseptic connectors. The process may proceed as follows, for example: a) Inside a biological safety cabinet (BSC), a suspension of cells from a biopsy that has been enzymatically digested is introduced into the “pre-growth chamber” (small unit on top of the cell tower), which is already filled with Initiation Growth Media containing antibiotics. From the BSC, the disposable would be transferred to the permanent ACE unit already in place; b) After approximately three days, the cells within the pre-growth chamber are trypsinized and introduced into the cell tower itself, which is pre-filled with Complete Growth Media. Here, the “bubbling action” caused by CO₂ injection force the media to circulate at such a rate that the cells spiral downward and settle on the surface of the discs in an evenly distributed manner; c) For approximately seven days, the cells are allowed to multiply. At this time, confluence may be checked to verify that culture is growing. Also at this time, the Complete Growth Media may be replaced with fresh Complete Growth Media. CGM will be replaced every seven days for three to four weeks. At the end of the culture period, the confluence is checked once more to verify that there is sufficient growth to possibly yield the desired quantity of cells for the intended treatment; d) If the culture is sufficiently confluent, it is harvested. The spent media (supernatant) is drained from the vessel. PBS will then is pumped into the vessel (to wash the media, FBS from the cells) and drained almost immediately. Trypsin-EDTA is pumped into the vessel to detach the cells from the growth surface. The trypsin/cell mixture is drained from the vessel and enter the spin separate. Cryopreservative is pumped into the vessel to rinse any residual cells from the surface of the discs, and be sent to the spin separator as well. The spin separator collects the cells and then evenly resuspend the cells in the shipping/injection medium. From the spin separator, the cells will be sent through an inline automated cell counting device or a sample collected for cell count and viability testing via laboratory analyses. Once a specific number of cells has been counted and the proper cell concentration has been reached, the harvested cells are delivered to a collection vial that can be removed to aliquot the samples for cryogenic freezing.

In another embodiment, automated robotic systems may be used to perform cell feeding, passaging, and harvesting for the entire length or a portion of the process. Cells can be introduced into the robotic device directly after digest and seed into the T-175 flask (or alternative). The device may have the capacity to incubate cells, perform cell count and viability analysis and perform feeds and transfers to larger culture vessels. The system may also have a computerized cataloging function to track individual lots. Existing technologies or customized systems may be used for the robotic option.

Growth conditions for cells encompassed herein comprise a temperature of approximately 30° C., 31° C., 32° C., 33° C., 34° C., 35° C., 36° C., 37° C., 38° C., 39° C., or 40° C. in a standard atmosphere comprising approximately 1%, 2%, 3%, 4%, 5%, 6%, 7%, 8%, 9%, 10% CO₂. Relative humidity is maintained at about 60%, 70%, 80%, 90%, 100%, or any ranger derivable therein. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

In particular embodiments, a non-limiting example medium for the culturing of the cells of the disclosure comprises Dulbecco’s Modified Essential Media (at times abbreviated DMEM herein). In some embodiments, DMEM-low glucose (also DMEM-LG herein) (Invitrogen, Carlsbad, Calif.) is utilized. The DMEM-low glucose may be supplemented, for example with approximately 5%, 10%, 15%, 20%, or any range derivable therein (v/v) fetal bovine serum (e.g. defined fetal bovine serum, Hyclone, Logan Utah), antibiotics/antimycotics (including for example penicillin (at approximately 10, 20, 30, 40, 50, 60, 70, 80, 90, 100, 125, 150, 175, 200, or any range derivable therein U/mL), streptomycin (at approximately 10, 20, 30, 40, 50, 60, 70, 80, 90, 100, 125, 150, 175, 200, or any range derivable therein mg/mL), and amphotericin B (at approximately 0.1, 0.25, 0.5, 0.75, 1.0, or any range derivable therein µg/mL), (Invitrogen, Carlsbad, Calif.)), and/or approximately 0.0001%, 0.001%, 0.01%, or any range derivable therein (v/v) 2-mercaptoethanol (Sigma, St. Louis Mo.). In some embodiments, different growth media are used, or different supplementations are provided, and these are normally indicated in the disclosure as supplementations to Growth Medium. One skilled in the art would recognize that modifications may be made to the media or supplements or supplementations that would also be suitable for practicing embodiments encompassed herein, which may also be used in embodiments encompassed herein.

Certain embodiments concern methods that provide cells that require no exogenous growth factors, except those available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Cells encompassed in some embodiments are capable of growth and expansion in chemically-defined growth media with no serum added. In such cases, the cells may require certain growth factors, which can be added to the medium to support and sustain the cells. Certain factors to be added for growth on serum-free media include one or more of FGF, EGF, IGF, and PDGF. In particular embodiments, two, three or all four of the factors are add to serum free or chemically defined media. In some embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer. Methods are provided wherein the cells can undergo at least 25, 30, 35, or 40 doublings prior to reaching a senescent state. Methods for deriving cells capable of doubling to reach 10¹⁴ cells or more are provided. Preferred are those methods which derive cells that can double sufficiently to produce at least about 10¹⁴, 10¹⁵, 10¹⁶, or 10¹⁷ or more cells when seeded at from about 10³ to about 10⁶ cells/cm² in culture. Preferably these cell numbers are produced within 80, 70, or 60 days or less. In one embodiment, cord tissue mesenchymal stem cells are isolated and expanded, and possess one or more markers selected from the group consisting of CD10, CD13, CD44, CD73, CD90, CD141, PDGFr-alpha, HLA-A,B,C, and a combination thereof. In addition, the cells do not produce one or more of CD31, CD34, CD45, CD117, CD141, and/or HLA-DR,DP, DQ.

### VIII. Administration of Cells

Cells from 10 flasks may be detached at a time and MSCs were resuspended in approximately 40 ml of M199 + approximately 1% human serum albumin (HSA; American Red Cross, Washington DC, USA). MSCs harvested from each 10-flask set may be stored for up to 4 h at 4° C. and combined at the end of the harvest. A total of 2-10 x 10⁶ MSC/kg may be resuspended in M199 + approximately 1% HSA and centrifuged at approximately 460 g for about 10 min at about 20° C. Cell pellets may be resuspended in fresh M199 + approximately 1% HSA media and centrifuged at about 460 g for about 10 min at about 20° C. for one, two, or three additional times. Total harvest time was 2-4 h based on MSC yield per flask and the target dose. Harvested MSC may be cryopreserved in Cryocyte (Baxter, Deerfield, IL, USA) freezing bags using a rate controlled freezer at a final concentration of 10% DMSO (Research Industries, Salt Lake City, UT, USA) and 5% HSA. On the day of infusion (when the cells that are generated as part of the disclosure are infused) cryopreserved units are thawed in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h. In one embodiment, MSC are generated according to protocols previously utilized for treatment of patients utilizing bone marrow derived MSC. Specifically, bone marrow may be aspirated (such as 10-30 mL) under local anesthesia (with or without sedation) from the posterior iliac crest, collected into sodium heparin containing tubes and transferred to a Good Manufacturing Practices (GMP) clean room. Bone marrow cells may be washed with a washing solution such as Dulbecco’s phosphate-buffered saline (DPBS), RPMI, or PBS supplemented with autologous patient plasma and layered on to 25 mL of Percoll (1.073 g/ml) at a concentration of approximately 1-2 x 10⁷ cells/mL. Subsequently the cells may be centrifuged at 900 g for approximately 30 min or a time period and rotation speed sufficient to achieve separation of mononuclear cells from debris and erythrocytes. Said cells may then be washed with PBS and plated at a density of approximately 1 x 10⁶ cells per mL in 175 cm² tissue culture flasks in DMEM with 10% FCS with flasks subsequently being loaded with a minimum of 30 million bone marrow mononuclear cells. The MSCs may be allowed to adhere for 72 h followed by media changes every 3-4 days. Adherent cells may be removed with 0.05% trypsin-EDTA and replated at a density of approximately 1 x 10⁶ per 175 cm². Although doses may be determined by one of skill in the art, and are dependent on various patient characteristics, intravenous administration may be performed at concentrations ranging from 1-10 million MSC per kilogram, including a dose of approximately 2-5 million cells per kilogram.

In some embodiments, fibroblasts are co-cultured with MSCs and encapsulated. In some embodiments, fibroblasts together with MSCs are encapsulated within the same membrane. In embodiments in which the cells are to be removed following implantation, a relatively large size structure encapsulating many cells, such as within a single membrane, may provide a convenient means for retrieval. A wide variety of materials may be used in various embodiments for microencapsulation of stem cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers. Techniques for microencapsulation of cells that may be used for administration of fibroblasts and/or stem cells are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of fibroblasts and/or stem cells. Certain embodiments incorporate stem cells into a polymer, such as a biopolymer or synthetic polymer. Examples of biopolymers include, but are not limited to, fibronectin, fibrin, fibrinogen, thrombin, collagen, and proteoglycans. Other factors, such as the cytokines discussed herein, can also be incorporated into the polymer. In some embodiments of the disclosure, fibroblasts and/or stem cells may be incorporated in the interstices of a three-dimensional gel. A large polymer or gel may be, for example, surgically implanted. A polymer or gel that can be formulated in small enough particles or fibers can be administered by other common, more convenient, non-surgical routes, for example.

### IX. Examples

### Example 1

### Culturing Immature Dendritic Cells With Fibroblasts

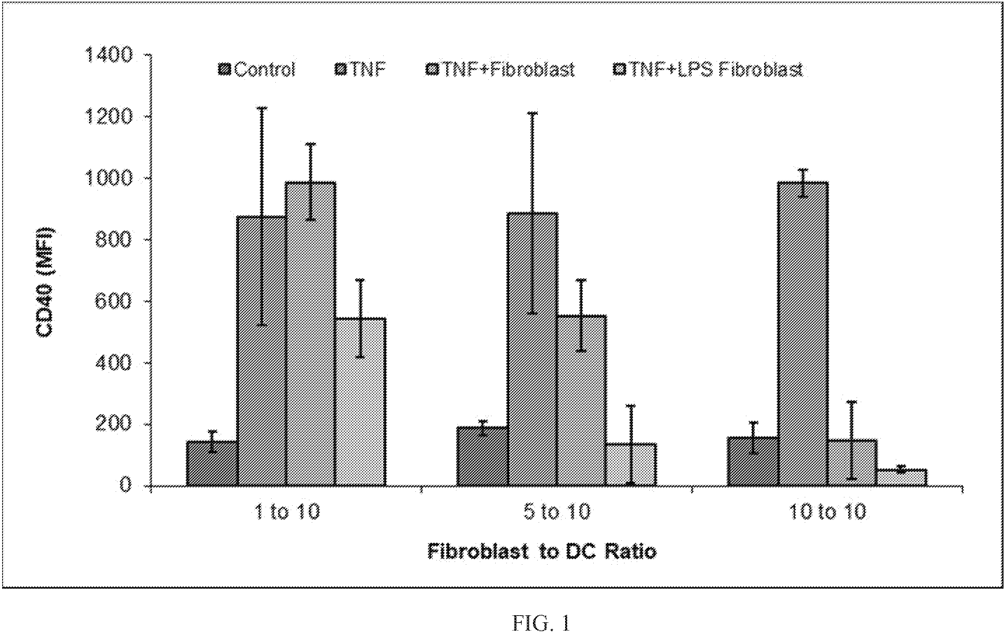

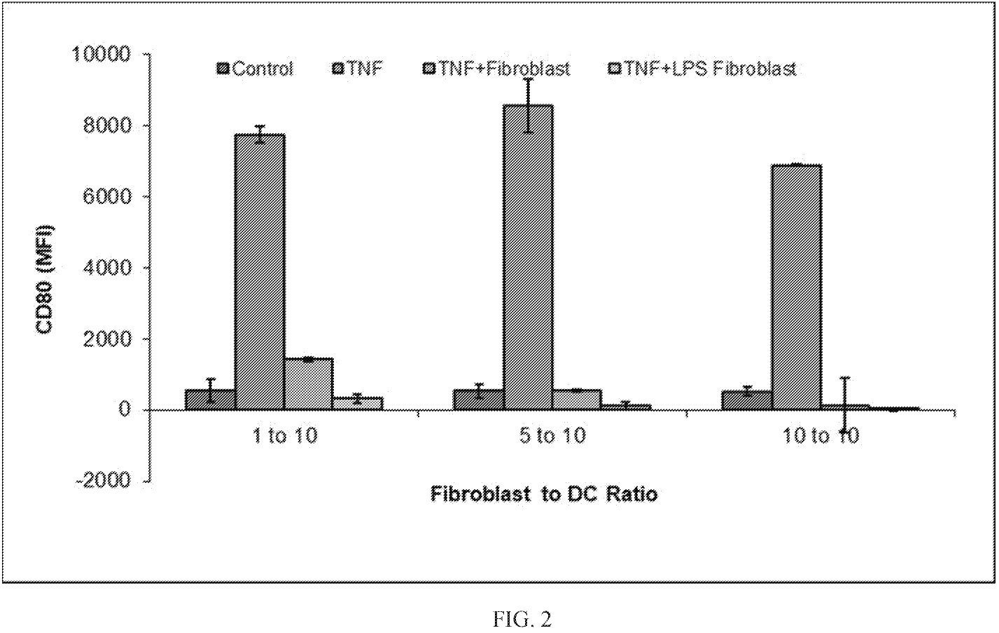

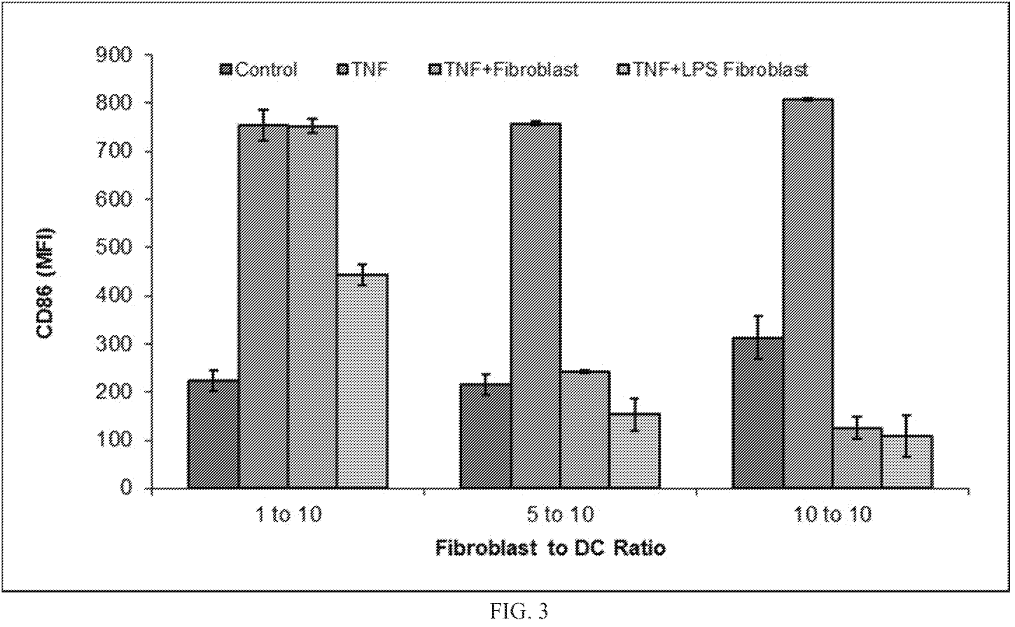

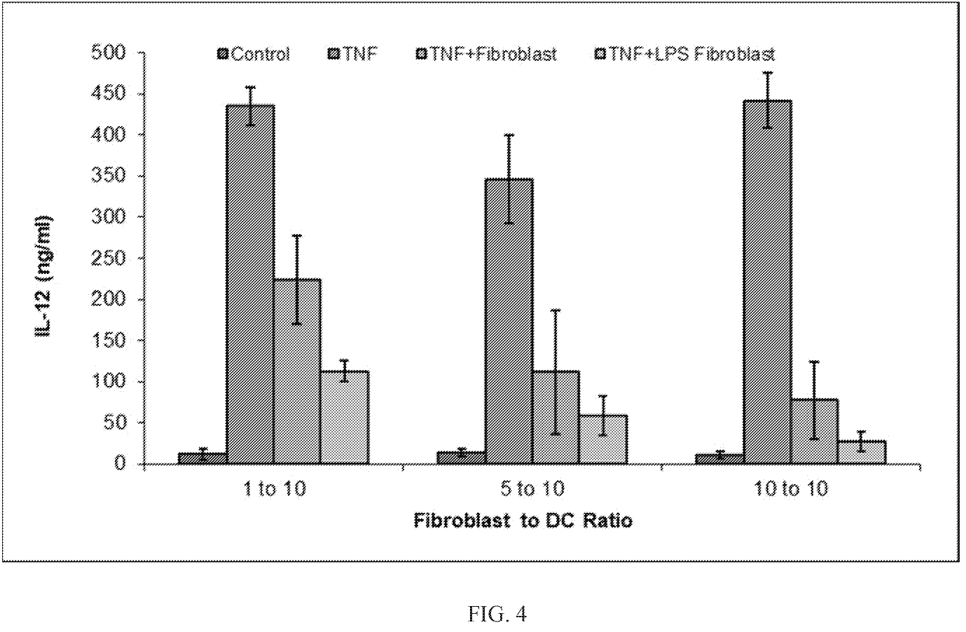

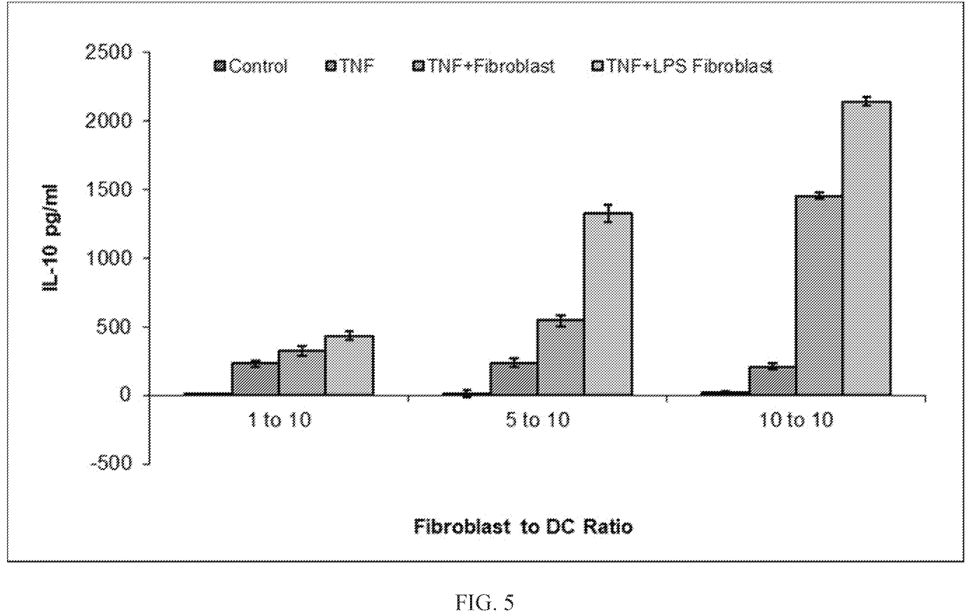

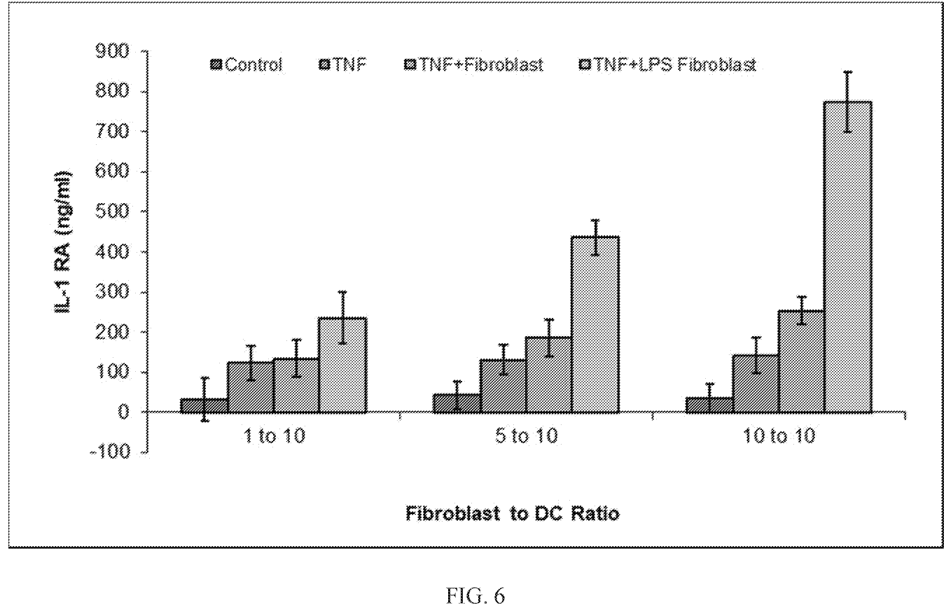

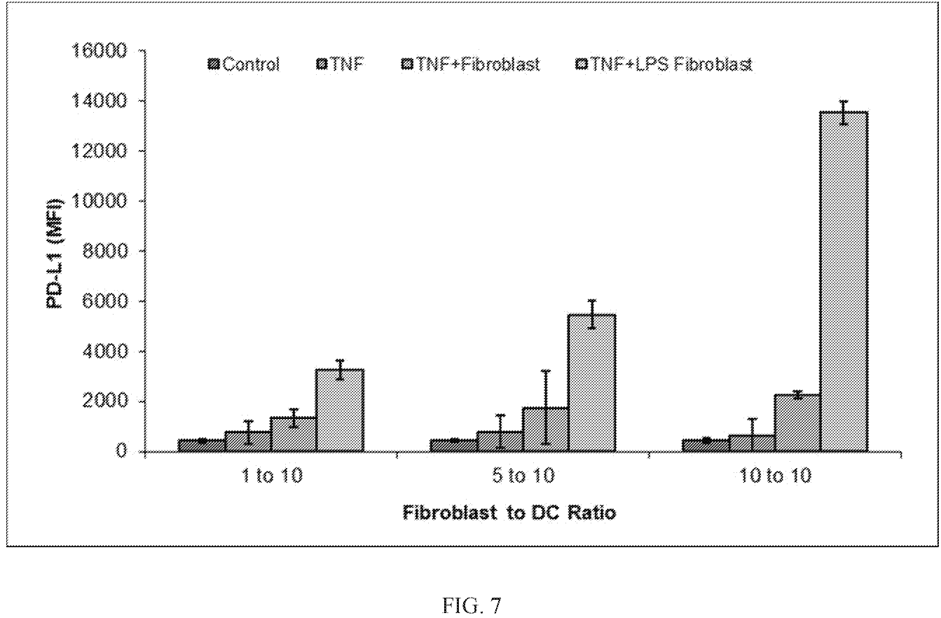

Dermal fibroblasts were obtained from ATCC and maintained in DMEM media with 10% FCS in a fully humidified environment, with penicillin/streptomycin mixture and non-essential amino acids. Cells were harvested at 75% confluence by trypsinization and plated with immature dendritic cells at day 5 of DC maturation. Cells were plated in 12 well plates with 100,000 fibroblasts per 1,000,000 DC, 500,000 fibroblasts per 1,000,000 DC and 1,000,000 fibroblasts per 1,000,000 DC. After 48 hours of culture, cells were extracted and CD40 (FIG. 1), CD80 (FIG. 2), CD86 (FIG. 3), and IL-12 (FIG. 4) expression was assessed by flow cytometry. After 48 hours of culture, cells were extracted and IL-10 production (FIG. 5), IL-1 RA production (FIG. 6), and PD-L1 expression by dendritic cells (FIG. 7) was assessed by ELISA.

Generation of DC was performed by culturing monocytes in GM-CSF and IL-4 for 5 days according to the method of Inaba et al (J Exp Med. 1992 Dec 1;176(6):1693-702) and subsequently matured by addition of TNF-alpha (where control is no TNF-alpha) on day 5 before the coculture. In some cultures LPS was added to the fibroblasts at a concentration of 5 µg/mL.

### Example 2

### Culturing Immature Dendritic Cells With Fibroblasts or MSCS

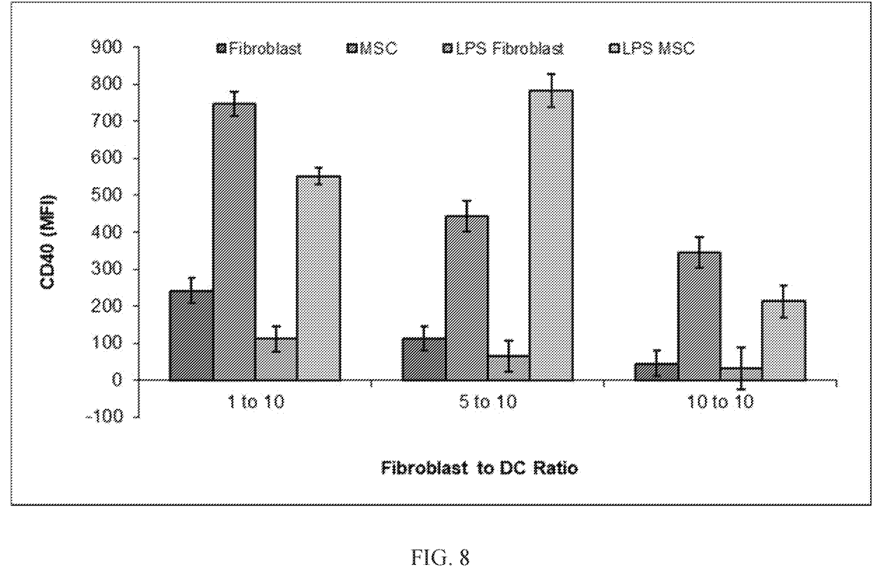

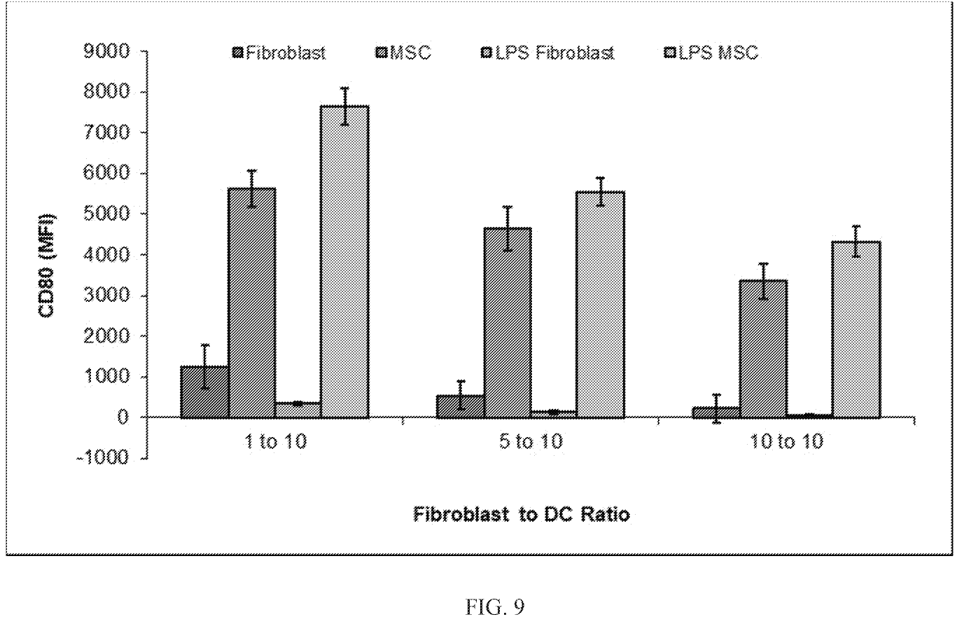

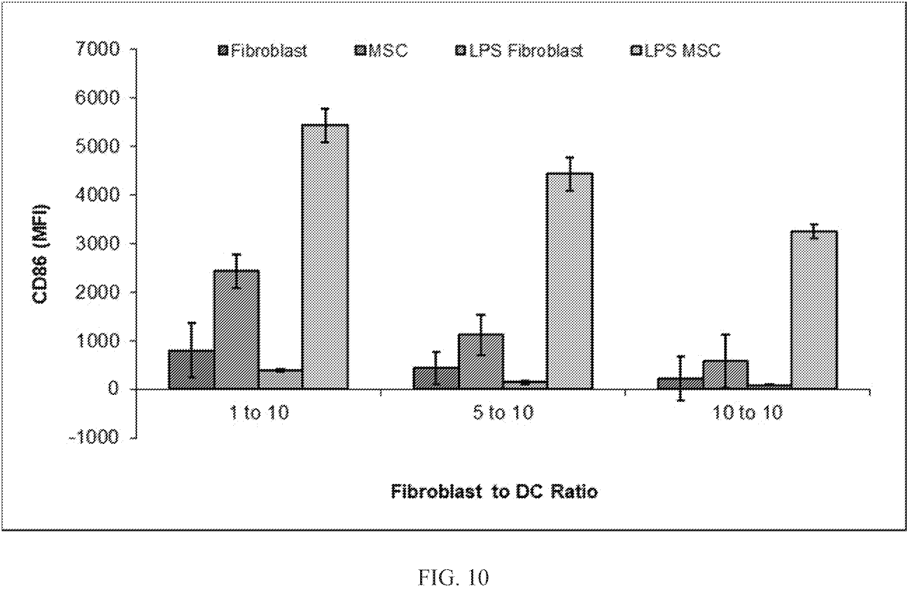

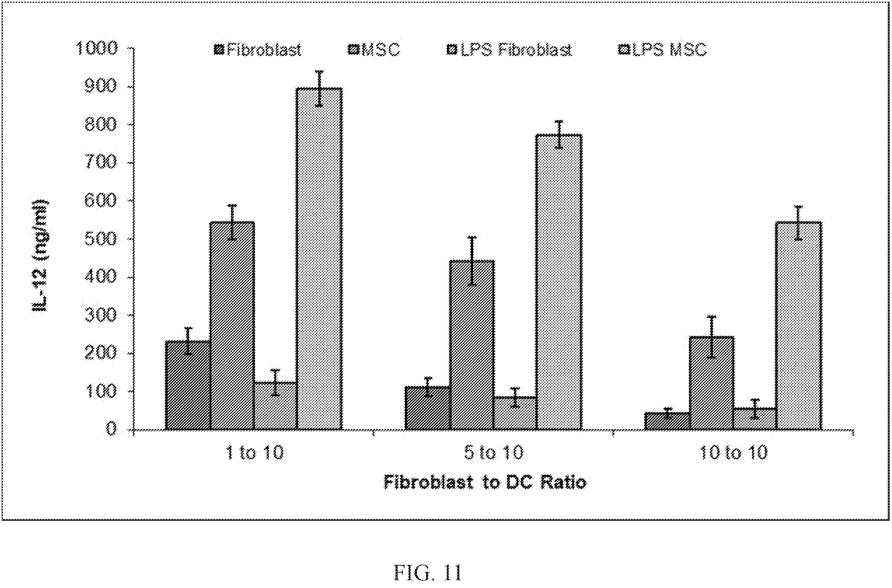

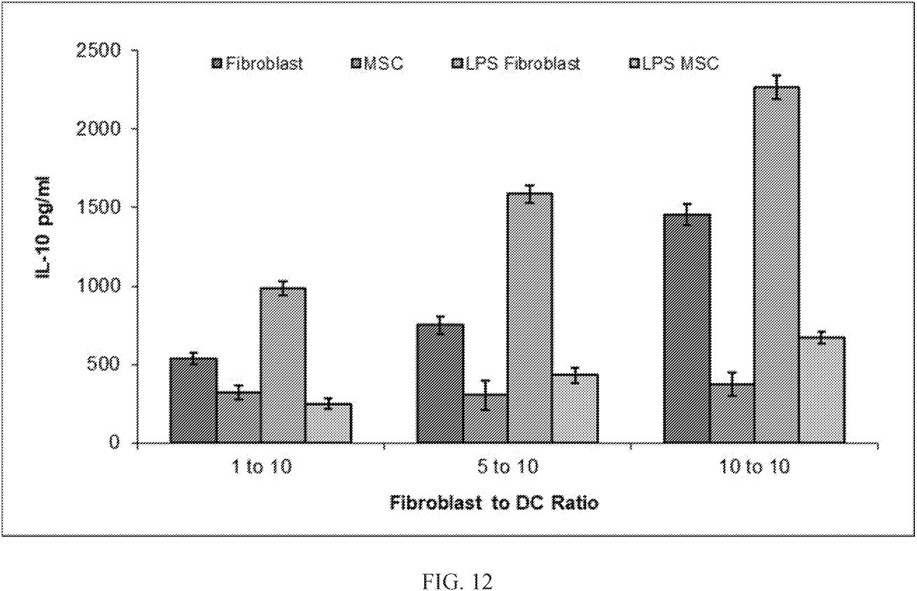

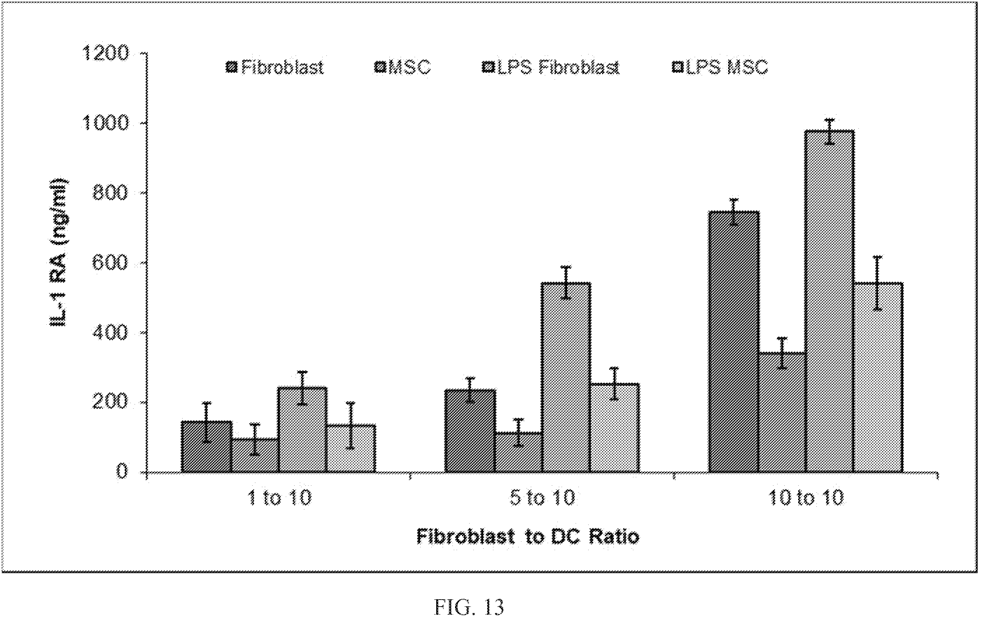

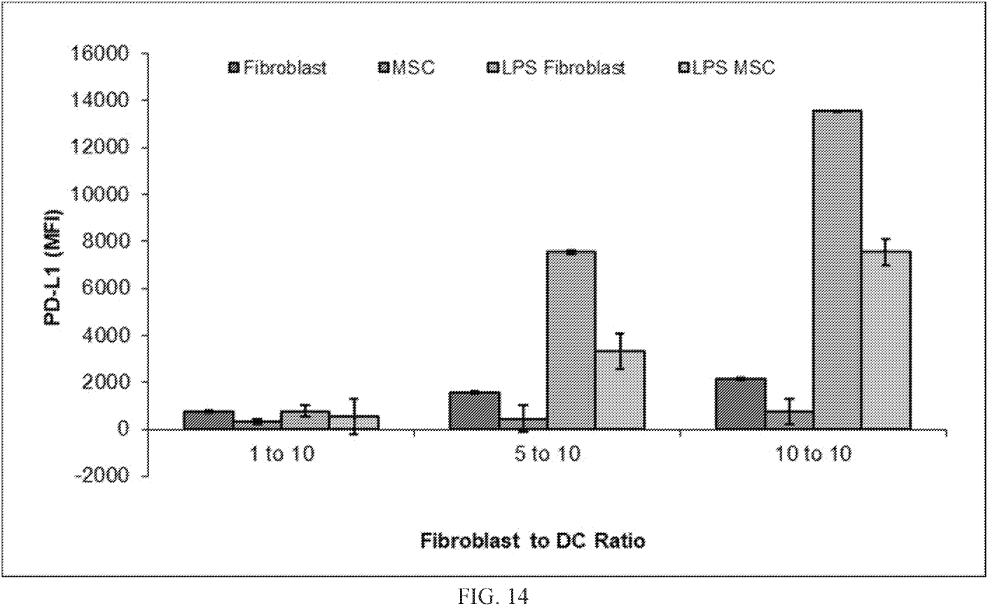

Dermal fibroblasts and bone marrow MSC were obtained from ATCC and maintained in DMEM media with 10% FCS in a fully humidified environment, with penicillin/streptomycin mixture and non-essential amino acids. Cells were harvested at 75% confluence by trypsinization and plated with immature dendritic cells at day 5 of DC maturation. Cells were plated in 12 well plates with 100,000 fibroblasts per 1,000,000 DC, 500,000 fibroblasts per 1,000,000 DC and 1,000,000 fibroblasts per 1,000,000 DC. MSC were also plated at the same concentrations. After 48 hours of culture, cells were extracted and CD40 (FIG. 8), CD80 (FIG. 9), CD86 (FIG. 10), and IL-12 (FIG. 11) expression on DC was assessed by flow cytometry. After 48 hours of culture, cells were extracted and IL-10 production (FIG. 12), IL-1 RA production (FIG. 13), and PD-L1 expression (FIG. 14) by dendritic cells was assessed by ELISA.

Generation of DC was performed by culturing monocytes in GM-CSF and IL-4 for 5 days according to the method of Inaba et al and subsequently matured by addition of TNF-alpha on day 5 before the coculture. In some cultures LPS was added to the fibroblasts at a concentration of 5 µg/mL.

### X. References

1. Croft, M., Duncan, D. D., and Swain, S. L. (1992) Response of naive antigen-specific CD4+ T cells in vitro: characteristics and antigen-presenting cell requirements. J Exp Med 176, 1431-1437

2. Knight, S. C., and Stagg, A. J. (1993) Antigen-presenting cell types. Curr Opin Immunol 5, 374-382

3. Morris, S. C., Lees, A., and Finkelman, F. D. (1994) In vivo activation of naive T cells by antigen-presenting B cells. J Immunol 152, 3777-3785

4. Constant, S., Sant’Angelo, D., Pasqualini, T., Taylor, T., Levin, D., Flavell, R., and Bottomly, K. (1995) Peptide and protein antigens require distinct antigen-presenting cell subsets for the priming of CD4+ T cells. J Immunol 154, 4915-4923

5. Di Nicola, M., Anichini, A., Mortarini, R., Bregni, M., Parmiani, G., and Gianni, A. M. (1998) Human dendritic cells: natural adjuvants in antitumor immunotherapy. Cytokines Cell Mol Ther 4, 265-273

6. Croft, M. (1994) Activation of naive, memory and effector T cells. Curr Opin Immunol 6, 431-437

7. Stem, A. S., Magram, J., and Presky, D. H. (1996) Interleukin-12 an integral cytokine in the immune response. Life Sci 58, 639-654

8. Naito, K., Inaba, K., Hirayama, Y., Inaba-Miyama, M., Sudo, T., and Muramatsu, S. (1989) Macrophage factors which enhance the mixed leukocyte reaction initiated by dendritic cells. J Immunol 142, 1834-1839

9. Wculek, S. K., Khouili, S. C., Priego, E., Heras-Murillo, I., and Sancho, D. (2019) Metabolic Control of Dendritic Cell Functions: Digesting Information. Front Immunol 10, 775

10. Kumar, S., Jeong, Y., Ashraf, M. U., and Bae, Y. S. (2019) Dendritic Cell-Mediated Th2 Immunity and Immune Disorders. Int J Mol Sci 20

11. Lenschow, D. J., Sperling, A. I., Cooke, M. P., Freeman, G., Rhee, L., Decker, D. C., Gray, G., Nadler, L. M., Goodnow, C. C., and Bluestone, J. A. (1994) Differential upregulation of the B7-1 and B7-2 costimulatory molecules after Ig receptor engagement by antigen. J Immunol 153, 1990-1997

12. Takauji, R., Iho, S., Takatsuka, H., Yamamoto, S., Takahashi, T., Kitagawa, H., Iwasaki, H., Iida, R., Yokochi, T., and Matsuki, T. (2002) CpG-DNA-induced IFN-alpha production involves p38 MAPK-dependent STAT1 phosphorylation in human plasmacytoid dendritic cell precursors. J Leukoc Biol 72, 1011-1019

13. Liu, Y. J. (2002) Uncover the mystery of plasmacytoid dendritic cell precursors or type 1 interferon producing cells by serendipity. Hum Immunol 63, 1067-1071

14. Bjorck, P. (2002) The multifaceted murine plasmacytoid dendritic cell. Hum Immunol 63, 1094-1102

15. Kadowaki, N., and Liu, Y. J. (2002) Natural type I interferon-producing cells as a link between innate and adaptive immunity. Hum Immunol 63, 1126-1132

16. Kemp, T. J., Elzey, B. D., and Griffith, T. S. (2003) Plasmacytoid dendritic cell-derived IFN-alpha induces TNF-related apoptosis-inducing ligand/Apo-2L-mediated antitumor activity by human monocytes following CpG oligodeoxynucleotide stimulation. J Immunol 171, 212-218

17. Ronnblom, L., Eloranta, M. L., and Alm, G. V. (2003) Role of natural interferon-alpha producing cells (plasmacytoid dendritic cells) in autoimmunity. Autoimmunity 36, 463-472

18. Vollenweider, R., and Lennert, K. (1983) Plasmacytoid T-cell clusters in nonspecific lymphadenitis. Virchows Arch B Cell Pathol Incl Mol Pathol 44, 1-14

19. Brado, B., and Moller, P. (1990) The plasmacytoid T cell or plasmacytoid monocyte--a sessile lymphoid cell with unique immunophenotype and unknown function, still awaiting lineage affiliation. Curr Top Pathol 84 ( Pt 1), 179-192

20. Facchetti, F., De Wolf-Peeters, C., Kennes, C., Rossi, G., De Vos, R., van den Oord, J. J., and Desmet, V. J. (1990) Leukemia-associated lymph node infiltrates of plasmacytoid monocytes (so-called plasmacytoid T-cells). Evidence for two distinct histological and immunophenotypical patterns. Am J Surg Pathol 14, 101-112

21. Koo, C. H., Mason, D. Y., Miller, R., Ben-Ezra, J., Sheibani, K., and Rappaport, H. (1990) Additional evidence that “plasmacytoid T-cell lymphoma” associated with chronic myeloproliferative disorders is of macrophage/monocyte origin. Am J Clin Pathol 93, 822-827

22. Caldwell, C. W., Yesus, Y. W., Loy, T. S., Bickel, J. T., and Perry, M. C. (1990) Acute leukemia/lymphoma of plasmacytoid T-cell type. Am J Clin Pathol 94, 778-786

23. Thomas, J. O., Beiske, K., Hann, I., Koo, C., and Mason, D. Y. (1991) Immunohistological diagnosis of “plasmacytoid T cell lymphoma” in paraffin wax sections. J Clin Pathol 44, 632-635

24. Baddoura, F. K., Hanson, C., and Chan, W. C. (1992) Plasmacytoid monocyte proliferation associated with myeloproliferative disorders. Cancer 69, 1457-1467

25. Facchetti, F., De Wolf-Peeters, C., De Vos, R., van den Oord, J. J., Pulford, K. A., and Desmet, V. J. (1989) Plasmacytoid monocytes (so-called plasmacytoid T cells) in granulomatous lymphadenitis. Hum Pathol 20, 588-593

26. Perussia, B., Fanning, V., and Trinchieri, G. (1985) A leukocyte subset bearing HLA-DR antigens is responsible for in vitro alpha interferon production in response to viruses. Nat Immun Cell Growth Regul 4, 120-137

27. Fitzgerald-Bocarsly, P., Feldman, M., Mendelsohn, M., Curl, S., and Lopez, C. (1988) Human mononuclear cells which produce interferon-alpha during NK(HSV-FS) assays are HLA-DR positive cells distinct from cytolytic natural killer effectors. J Leukoc Biol 43, 323-334

28. Feldman, M., and Fitzgerald-Bocarsly, P. (1990) Sequential enrichment and immunocytochemical visualization of human interferon-alpha-producing cells. J Interferon Res 10, 435-446

29. Colonna, M., Trinchieri, G., and Liu, Y. J. (2004) Plasmacytoid dendritic cells in immunity. Nat Immunol 5, 1219-1226

30. Wang, Y. H., and Liu, Y. J. (2004) Mysterious origin of plasmacytoid dendritic cell precursors. Immunity 21, 1-2

31. Gary-Gouy, H., Lebon, P., and Dalloul, A. H. (2002) Type I interferon production by plasmacytoid dendritic cells and monocytes is triggered by viruses, but the level of production is controlled by distinct cytokines. J Interferon Cytokine Res 22, 653-659

32. Pichyangkul, S., Endy, T. P., Kalayanarooj, S., Nisalak, A., Yongvanitchit, K., Green, S., Rothman, A. L., Ennis, F. A., and Libraty, D. H. (2003) A blunted blood plasmacytoid dendritic cell response to an acute systemic viral infection is associated with increased disease severity. J Immunol 171, 5571-5578

33. Hughes, C. C., Savage, C. O., and Pober, J. S. (1990) The endothelial cell as a regulator of T-cell function. Immunol Rev 117, 85-102

34. Pober, J. S., and Cotran, R. S. (1991) Immunologic interactions of T lymphocytes with vascular endothelium. Adv Immunol 50, 261-302

35. Pulendran, B., Banchereau, J., Burkeholder, S., Kraus, E., Guinet, E., Chalouni, C., Caron, D., Maliszewski, C., Davoust, J., Fay, J., and Palucka, K. (2000) Flt3-ligand and granulocyte colony-stimulating factor mobilize distinct human dendritic cell subsets in vivo. J Immunol 165, 566-572

36. Ratta, M., Rondelli, D., Fortuna, A., Curti, A., Fogli, M., Fagnoni, F., Martinelli, G., Terragna, C., Tura, S., and Lemoli, R. M. (1998) Generation and functional characterization of human dendritic cells derived from CD34 cells mobilized into peripheral blood: comparison with bone marrow CD34+ cells. Br J Haematol 101, 756-765

37. Wei, S., Kryczek, I., Zou, L., Daniel, B., Cheng, P., Mottram, P., Curiel, T., Lange, A., and Zou, W. (2005) Plasmacytoid dendritic cells induce CD8+ regulatory T cells in human ovarian carcinoma. Cancer Res 65, 5020-5026

38. Fiebich, B. L., Mueksch, B., Boehringer, M., and Hull, M. (2000) Interleukin-1beta induces cyclooxygenase-2 and prostaglandin E(2) synthesis in human neuroblastoma cells: involvement of p38 mitogen-activated protein kinase and nuclear factor-kappaB. J Neurochem 75, 2020-2028

39. Mohammed, S. I., Coffman, K., Glickman, N. W., Hayek, M. G., Waters, D. J., Schlittler, D., DeNicola, D. B., and Knapp, D. W. (2001) Prostaglandin E2 concentrations in naturally occurring canine cancer. Prostaglandins Leukot Essent Fatty Acids 64, 1-4

40. Casibang, M., Purdom, S., Jakowlew, S., Neckers, L., Zia, F., Ben-Av, P., Hla, T., You, L., Jablons, D. M., and Moody, T. W. (2001) Prostaglandin E2 and vasoactive intestinal peptide increase vascular endothelial cell growth factor mRNAs in lung cancer cells. Lung Cancer 31, 203-212

41. Chen, W. S., Wei, S. J., Liu, J. M., Hsiao, M., Kou-Lin, J., and Yang, W. K. (2001) Tumor invasiveness and liver metastasis of colon cancer cells correlated with cyclooxygenase-2 (COX-2) expression and inhibited by a COX-2-selective inhibitor, etodolac. Int J Cancer 91, 894-899

42. Sheng, H., Shao, J., Washington, M. K., and DuBois, R. N. (2001) Prostaglandin E2 increases growth and motility of colorectal carcinoma cells. J Biol Chem 276, 18075-18081

43. Pai, R., Szabo, I. L., Soreghan, B. A., Atay, S., Kawanaka, H., and Tarnawski, A. S. (2001) PGE(2) stimulates VEGF expression in endothelial cells via ERK2/JNK1 signaling pathways. Biochem Biophys Res Commun 286, 923-928

44. Kawamori, T., Uchiya, N., Nakatsugi, S., Watanabe, K., Ohuchida, S., Yamamoto, H., Maruyama, T., Kondo, K., Sugimura, T., and Wakabayashi, K. (2001) Chemopreventive effects of ONO-8711, a selective prostaglandin E receptor EP(1) antagonist, on breast cancer development. Carcinogenesis 22, 2001-2004

45. Denkert, C., Kobel, M., Pest, S., Koch, I., Berger, S., Schwabe, M., Siegert, A., Reles, A., Klosterhalfen, B., and Hauptmann, S. (2002) Expression of cyclooxygenase 2 is an independent prognostic factor in human ovarian carcinoma. Am J Pathol 160, 893-903

46. Luft, T., Jefford, M., Luetjens, P., Toy, T., Hochrein, H., Masterman, K. A., Maliszewski, C., Shortman, K., Cebon, J., and Maraskovsky, E. (2002) Functionally distinct dendritic cell (DC) populations induced by physiologic stimuli: prostaglandin E(2) regulates the migratory capacity of specific DC subsets. Blood 100, 1362-1372

47. Hidalgo, G. E., Zhong, L., Doherty, D. E., and Hirschowitz, E. A. (2002) Plasma PGE-2 levels and altered cytokine profiles in adherent peripheral blood mononuclear cells in non-small cell lung cancer (NSCLC). Mol Cancer 1, 5

48. Yang, L., Yamagata, N., Yadav, R., Brandon, S., Courtney, R. L., Morrow, J. D., Shyr, Y., Boothby, M., Joyce, S., Carbone, D. P., and Breyer, R. M. (2003) Cancer-associated immunodeficiency and dendritic cell abnormalities mediated by the prostaglandin EP2 receptor. J Clin Invest 111, 727-735

49. Yakar, I., Melamed, R., Shakhar, G., Shakhar, K., Rosenne, E., Abudarham, N., Page, G. G., and Ben-Eliyahu, S. (2003) Prostaglandin e(2) suppresses NK activity in vivo and promotes postoperative tumor metastasis in rats. Ann Surg Oncol 10, 469-479

50. Eibl, G., Bruemmer, D., Okada, Y., Duffy, J. P., Law, R. E., Reber, H. A., and Hines, O. J. (2003) PGE(2) is generated by specific COX-2 activity and increases VEGF production in COX-2-expressing human pancreatic cancer cells. Biochem Biophys Res Commun 306, 887-897

51. Shao, J., Lee, S. B., Guo, H., Evers, B. M., and Sheng, H. (2003) Prostaglandin E2 stimulates the growth of colon cancer cells via induction of amphiregulin. Cancer Res 63, 5218-5223

52. Sandler, A. B., and Dubinett, S. M. (2004) COX-2 inhibition and lung cancer. Semin Oncol 31, 45-52

53. Zhu, G., Saed, G. M., Deppe, G., Diamond, M. P., and Munkarah, A. R. (2004) Hypoxia up-regulates the effects of prostaglandin E2 on tumor angiogenesis in ovarian cancer cells. Gynecol Oncol 94, 422-426

54. Akasaki, Y., Liu, G., Chung, N. H., Ehtesham, M., Black, K. L., and Yu, J. S. (2004) Induction of a CD4+ T regulatory type 1 response by cyclooxygenase-2-overexpressing glioma. J Immunol 173, 4352-4359

55. Cianchi, F., Cortesini, C., Schiavone, N., Perna, F., Magnelli, L., Fanti, E., Bani, D., Messerini, L., Fabbroni, V., Perigli, G., Capaccioli, S., and Masini, E. (2005) The role of cyclooxygenase-2 in mediating the effects of histamine on cell proliferation and vascular endothelial growth factor production in colorectal cancer. Clin Cancer Res 11, 6807-6815

56. Chemnitz, J. M., Driesen, J., Classen, S., Riley, J. L., Debey, S., Beyer, M., Popov, A., Zander, T., and Schultze, J. L. (2006) Prostaglandin E2 impairs CD4+ T cell activation by inhibition of lck: implications in Hodgkin’s lymphoma. Cancer Res 66, 1114-1122

57. Cui, X., Yang, S. C., Sharma, S., Heuze-Vourc’h, N., and Dubinett, S. M. (2006) IL-4 regulates COX-2 and PGE2 production in human non-small cell lung cancer. Biochem Biophys Res Commun 343, 995-1001

58. Backlund, M. G., Mann, J. R., Wang, D., and Dubois, R. N. (2006) Ras upregulation of cyclooxygenase-2. Methods Enzymol 407, 401-410

59. Alder, J., Hahn-Zoric, M., Andersson, B. A., and Karlsson-Parra, A. (2006) Interferon-gamma dose-dependently inhibits prostaglandin E2-mediated dendritic-cell-migration towards secondary lymphoid organ chemokines. Vaccine 24, 7087-7094

60. Bergmann, C., Strauss, L., Zeidler, R., Lang, S., and Whiteside, T. L. (2007) Expansion of human T regulatory type 1 cells in the microenvironment of cyclooxygenase 2 overexpressing head and neck squamous cell carcinoma. Cancer Res 67, 8865-8873

61. Yaqub, S., Henjum, K., Mahic, M., Jahnsen, F. L., Aandahl, E. M., Bjornbeth, B. A., and Tasken, K. (2008) Regulatory T cells in colorectal cancer patients suppress antitumor immune activity in a COX-2 dependent manner. Cancer Immunol Immunother 57, 813-821

62. Jarnicki, A. G., Conroy, H., Brereton, C., Donnelly, G., Toomey, D., Walsh, K., Sweeney, C., Leavy, O., Fletcher, J., Lavelle, E. C., Dunne, P., and Mills, K. H. (2008) Attenuating regulatory T cell induction by TLR agonists through inhibition of p38 MAPK signaling in dendritic cells enhances their efficacy as vaccine adjuvants and cancer immunotherapeutics. J Immunol 180, 3797-3806

63. Sun, Q., Liu, Q., Zheng, Y., and Cao, X. (2008) Rapamycin suppresses TLR4-triggered IL-6 and PGE(2) production of colon cancer cells by inhibiting TLR4 expression and NF-kappaB activation. Mol Immunol 45, 2929-2936

64. O’Callaghan, G., Kelly, J., Shanahan, F., and Houston, A. (2008) Prostaglandin E2 stimulates Fas ligand expression via the EP1 receptor in colon cancer cells. Br J Cancer 99, 502-512

65. Baryawno, N., Sveinbjornsson, B., Eksborg, S., Orrego, A., Segerstrom, L., Oqvist, C. O., Holm, S., Gustavsson, B., Kagedal, B., Kogner, P., and Johnsen, J. I. (2008) Tumor-growth-promoting cyclooxygenase-2 prostaglandin E2 pathway provides medulloblastoma therapeutic targets. Neuro Oncol 10, 661-674

66. Greenhough, A., Smartt, H. J., Moore, A. E., Roberts, H. R., Williams, A. C., Paraskeva, C., and Kaidi, A. (2009) The COX-2/PGE2 pathway: key roles in the hallmarks of cancer and adaptation to the tumour microenvironment. Carcinogenesis 30, 377-386

67. Zhang, Y., Liu, Q., Zhang, M., Yu, Y., Liu, X., and Cao, X. (2009) Fas signal promotes lung cancer growth by recruiting myeloid-derived suppressor cells via cancer cell-derived PGE2. J Immunol 182, 3801-3808

68. Bluth, M. J., Zaba, L. C., Moussai, D., Suarez-Farinas, M., Kaporis, H., Fan, L., Pierson, K. C., White, T. R., Pitts-Kiefer, A., Fuentes-Duculan, J., Guttman-Yassky, E., Krueger, J. G., Lowes, M. A., and Carucci, J. A. (2009) Myeloid dendritic cells from human cutaneous squamous cell carcinoma are poor stimulators of T-cell proliferation. J Invest Dermatol 129, 2451-2462

69. Liu, Q., Zhang, C., Sun, A., Zheng, Y., Wang, L., and Cao, X. (2009) Tumor-educated CD11bhighIalow regulatory dendritic cells suppress T cell response through arginase I. J Immunol 182, 6207-6216

70. Eruslanov, E., Kaliberov, S., Daurkin, I., Kaliberova, L., Buchsbaum, D., Vieweg, J., and Kusmartsev, S. (2009) Altered expression of 15-hydroxyprostaglandin dehydrogenase in tumor-infiltrated CD11b myeloid cells: a mechanism for immune evasion in cancer. J Immunol 182, 7548-7557

71. Chattopadhyay, S., Bhattacharyya, S., Saha, B., Chakraborty, J., Mohanty, S., Sakib Hossain, D. M., Banerjee, S., Das, K., Sa, G., and Das, T. (2009) Tumor-shed PGE(2) impairs IL2Rgammac-signaling to inhibit CD4 T cell survival: regulation by theaflavins. PLoS One 4, e7382

72. Balsamo, M., Scordamaglia, F., Pietra, G., Manzini, C., Cantoni, C., Boitano, M., Queirolo, P., Vermi, W., Facchetti, F., Moretta, A., Moretta, L., Mingari, M. C., and Vitale, M. (2009) Melanoma-associated fibroblasts modulate NK cell phenotype and antitumor cytotoxicity. Proc Natl Acad Sci U S A 106, 20847-20852

73. Mougiakakos, D., Johansson, C. C., Trocme, E., All-Ericsson, C., Economou, M. A., Larsson, O., Seregard, S., and Kiessling, R. (2010) Intratumoral forkhead box P3-positive regulatory T cells predict poor survival in cyclooxygenase-2-positive uveal melanoma. Cancer 116, 2224-2233

74. Muthuswamy, R., Mueller-Berghaus, J., Haberkom, U., Reinhart, T. A., Schadendorf, D., and Kalinski, P. (2010) PGE(2) transiently enhances DC expression of CCR7 but inhibits the ability of DCs to produce CCL19 and attract naive T cells. Blood 116, 1454-1459

75. Lechner, M. G., Liebertz, D. J., and Epstein, A. L. (2010) Characterization of cytokine-induced myeloid-derived suppressor cells from normal human peripheral blood mononuclear cells. J Immunol 185, 2273-2284

76. Sakata, D., Yao, C., and Narumiya, S. (2010) Emerging roles of prostanoids in T cell-mediated immunity. IUBMB Life 62, 591-596

77. Baratelli, F., Lee, J. M., Hazra, S., Lin, Y., Walser, T. C., Schaue, D., Pak, P. S., Elashoff, D., Reckamp, K., Zhang, L., Fishbein, M. C., Sharma, S., and Dubinett, S. M. (2010) PGE(2) contributes to TGF-beta induced T regulatory cell function in human non-small cell lung cancer. Am J Transl Res 2, 356-367

78. Su, Y., Jackson, E. K., and Gorelik, E. (2011) Receptor desensitization and blockade of the suppressive effects of prostaglandin E(2) and adenosine on the cytotoxic activity of human melanoma-infiltrating T lymphocytes. Cancer Immunol Immunother 60, 111-122

79. Oshima, H., Hioki, K., Popivanova, B. K., Oguma, K., Van Rooijen, N., Ishikawa, T. O., and Oshima, M. (2011) Prostaglandin E(2) signaling and bacterial infection recruit tumor-promoting macrophages to mouse gastric tumors. Gastroenterology 140, 596-607 e597

80. Qian, X., Zhang, J., and Liu, J. (2011) Tumor-secreted PGE2 inhibits CCL5 production in activated macrophages through cAMP/PKA signaling pathway. J Biol Chem 286, 2111-2120

81. Brecht, K., Weigert, A., Hu, J., Popp, R., Fisslthaler, B., Korff, T., Fleming, I., Geisslinger, G., and Brune, B. (2011) Macrophages programmed by apoptotic cells promote angiogenesis via prostaglandin E2. FASEB J 25, 2408-2417

82. Heusinkveld, M., de Vos van Steenwijk, P. J., Goedemans, R., Ramwadhdoebe, T. H., Gorter, A., Welters, M. J., van Hall, T., and van der Burg, S. H. (2011) M2 macrophages induced by prostaglandin E2 and IL-6 from cervical carcinoma are switched to activated M1 macrophages by CD4+ Th1 cells. J Immunol 187, 1157-1165

83. Chen, E. P., and Smyth, E. M. (2011) COX-2 and PGE2-dependent immunomodulation in breast cancer. Prostaglandins Other Lipid Mediat 96, 14-20

84. Obermajer, N., Muthuswamy, R., Lesnock, J., Edwards, R. P., and Kalinski, P. (2011) Positive feedback between PGE2 and COX2 redirects the differentiation of human dendritic cells toward stable myeloid-derived suppressor cells. Blood 118, 5498-5505

85. Whiteside, T. L. (2012) Disarming suppressor cells to improve immunotherapy. Cancer Immunol Immunother 61, 283-288

86. Kalinski, P. (2012) Regulation of immune responses by prostaglandin E2. J Immunol 188, 21-28

87. Lanzinger, M., Jurgens, B., Hainz, U., Dillinger, B., Raberger, J., Fuchs, D., and Heitger, A. (2012) Ambivalent effects of dendritic cells displaying prostaglandin E2-induced indoleamine 2,3-dioxygenase. Eur J Immunol 42, 1117-1128

88. Sha, W., Brune, B., and Weigert, A. (2012) The multi-faceted roles of prostaglandin E2 in cancer-infiltrating mononuclear phagocyte biology. Immunobiology 217, 1225-1232

89. Mandapathil, M., Szczepanski, M., Harasymczuk, M., Ren, J., Cheng, D., Jackson, E. K., Gorelik, E., Johnson, J., Lang, S., and Whiteside, T. L. (2012) CD26 expression and adenosine deaminase activity in regulatory T cells (Treg) and CD4(+) T effector cells in patients with head and neck squamous cell carcinoma. Oncoimmunology 1, 659-669

90. Obermajer, N., and Kalinski, P. (2012) Key role of the positive feedback between PGE(2) and COX2 in the biology of myeloid-derived suppressor cells. Oncoimmunology 1, 762-764

91. Obermajer, N., Wong, J. L., Edwards, R. P., Odunsi, K., Moysich, K., and Kalinski, P. (2012) PGE(2)-driven induction and maintenance of cancer-associated myeloid-derived suppressor cells. Immunol Invest 41, 635-657

92. Hou, W., Sampath, P., Rojas, J. J., and Thorne, S. H. (2016) Oncolytic Virus-Mediated Targeting of PGE2 in the Tumor Alters the Immune Status and Sensitizes Established and Resistant Tumors to Immunotherapy. Cancer Cell 30, 108-119

93. Moltu, K., Henjum, K., Oberprieler, N. G., Bjornbeth, B. A., and Tasken, K. (2017) Proximal signaling responses in peripheral T cells from colorectal cancer patients are affected by high concentrations of circulating prostaglandin E2. Hum Immunol 78, 129-137

94. Moreau, P., Carosella, E., Teyssier, M., Prost, S., Gluckman, E., Dausset, J., and Kirszenbaum, M. (1995) Soluble HLA-G molecule. An alternatively spliced HLA-G mRNA form candidate to encode it in peripheral blood mononuclear cells and human trophoblasts. Hum Immunol 43, 231-236

95. Rouas-Freiss, N., Khalil-Daher, I., Riteau, B., Menier, C., Paul, P., Dausset, J., and Carosella, E. D. (1999) The immunotolerance role of HLA-G. Semin Cancer Biol 9, 3-12

96. Rajagopalan, S., and Long, E. O. (1999) A human histocompatibility leukocyte antigen (HLA)-G-specific receptor expressed on all natural killer cells. J Exp Med 189, 1093-1100

97. Carosella, E. D., Dausset, J., and Rouas-Freiss, N. (1999) Immunotolerant functions of HLA-G. Cell Mol Life Sci 55, 327-333

98. Le Bouteiller, P., and Blaschitz, A. (1999) The functionality of HLA-G is emerging. Immunol Rev 167, 233-244

99. Fournel, S., Aguerre-Girr, M., Huc, X., Lenfant, F., Alam, A., Toubert, A., Bensussan, A., and Le Bouteiller, P. (2000) Cutting edge: soluble HLA-G1 triggers CD95/CD95 ligand-mediated apoptosis in activated CD8+ cells by interacting with CD8. J Immunol 164, 6100-6104

100. Ugurel, S., Rebmann, V., Ferrone, S., Tilgen, W., Grosse-Wilde, H., and Reinhold, U. (2001) Soluble human leukocyte antigen--G serum level is elevated in melanoma patients and is further increased by interferon-alpha immunotherapy. Cancer 92, 369-376

101. Lila, N., Rouas-Freiss, N., Dausset, J., Carpentier, A., and Carosella, E. D. (2001) Soluble HLA-G protein secreted by allo-specific CD4+ T cells suppresses the allo-proliferative response: a CD4+ T cell regulatory mechanism. Proc Natl Acad Sci U S A 98, 12150-12155

102. Wiendl, H., Mitsdoerffer, M., Hofmeister, V., Wischhusen, J., Bornemann, A., Meyermann, R., Weiss, E. H., Melms, A., and Weller, M. (2002) A functional role of HLA-G expression in human gliomas: an alternative strategy of immune escape. J Immunol 168, 4772-4780

103. Lila, N., Amrein, C., Guillemain, R., Chevalier, P., Latremouille, C., Fabiani, J. N., Dausset, J., Carosella, E. D., and Carpentier, A. (2002) Human leukocyte antigen-G expression after heart transplantation is associated with a reduced incidence of rejection. Circulation 105, 1949-1954

104. McIntire, R. H., Morales, P. J., Petroff, M. G., Colonna, M., and Hunt, J. S. (2004) Recombinant HLA-G5 and -G6 drive U937 myelomonocytic cell production of TGF-beta1. J Leukoc Biol 76, 1220-1228

105. Mitsdoerffer, M., Schreiner, B., Kieseier, B. C., Neuhaus, O., Dichgans, J., Hartung, H. P., Weller, M., and Wiendl, H. (2005) Monocyte-derived HLA-G acts as a strong inhibitor of autologous CD4 T cell activation and is upregulated by interferon-beta in vitro and in vivo: rationale for the therapy of multiple sclerosis. J Neuroimmunol 159, 155-164

106. Bahri, R., Hirsch, F., Josse, A., Rouas-Freiss, N., Bidere, N., Vasquez, A., Carosella, E. D., Charpentier, B., and Durrbach, A. (2006) Soluble HLA-G inhibits cell cycle progression in human alloreactive T lymphocytes. J Immunol 176, 1331-1339

107. Le Rond, S., Azema, C., Krawice-Radanne, I., Durrbach, A., Guettier, C., Carosella, E. D., and Rouas-Freiss, N. (2006) Evidence to support the role of HLA-G5 in allograft acceptance through induction of immunosuppressive/ regulatory T cells. J Immunol 176, 3266-3276

108. Lindaman, A., Dowden, A., and Zavazava, N. (2006) Soluble HLA-G molecules induce apoptosis in natural killer cells. Am J Reprod Immunol 56, 68-76

109. Naji, A., Durrbach, A., Carosella, E. D., and Rouas-Freiss, N. (2007) Soluble HLA-G and HLA-G1 expressing antigen-presenting cells inhibit T-cell alloproliferation through ILT-2/ILT-4/FasL-mediated pathways. Hum Immunol 68, 233-239

110. Gros, F., Cabillic, F., Toutirais, O., Maux, A. L., Sebti, Y., and Amiot, L. (2008) Soluble HLA-G molecules impair natural killer/dendritic cell crosstalk via inhibition of dendritic cells. Eur J Immunol 38, 742-749

111. Rizzo, R., Campioni, D., Stignani, M., Melchiorri, L., Bagnara, G. P., Bonsi, L., Alviano, F., Lanzoni, G., Moretti, S., Cuneo, A., Lanza, F., and Baricordi, O. R. (2008) A functional role for soluble HLA-G antigens in immune modulation mediated by mesenchymal stromal cells. Cytotherapy 10, 364-375

112. Lajoie, J., Fontaine, J., Tremblay, C., Routy, J. P., Poudrier, J., and Roger, M. (2009) Persistence of high levels of blood soluble human leukocyte antigen-G is associated with rapid progression of HIV infection. AIDS 23, 1437-1440

113. Liu, X. H., Kirschenbaum, A., Lu, M., Yao, S., Dosoretz, A., Holland, J. F., and Levine, A. C. (2002) Prostaglandin E2 induces hypoxia-inducible factor-1alpha stabilization and nuclear localization in a human prostate cancer cell line. J Biol Chem 277, 50081-50086

114. Fukuda, R., Kelly, B., and Semenza, G. L. (2003) Vascular endothelial growth factor gene expression in colon cancer cells exposed to prostaglandin E2 is mediated by hypoxia-inducible factor 1. Cancer Res 63, 2330-2334

115. Van Pham, P., Truong, N. C., Le, P. T., Tran, T. D., Vu, N. B., Bui, K. H., and Phan, N. K. (2015) Isolation and proliferation of umbilical cord tissue derived mesenchymal stem cells for clinical applications. Cell and tissue banking

116. Fazzina, R., Mariotti, A., Procoli, A., Fioravanti, D., Iudicone, P., Scambia, G., Pierelli, L., and Bonanno, G. (2015) A new standardized clinical-grade protocol for banking human umbilical cord tissue cells. Transfusion 55, 2864-2873

117. Bieback, K. (2013) Platelet lysate as replacement for fetal bovine serum in mesenchymal stromal cell cultures. Transfusion medicine and hemotherapy : offizielles Organ der Deutschen Gesellschaft fur Transfusionsmedizin und Immunhamatologie 40, 326-335

118. Stanko, P., Kaiserova, K., Altanerova, V., and Altaner, C. (2014) Comparison of human mesenchymal stem cells derived from dental pulp, bone marrow, adipose tissue, and umbilical cord tissue by gene expression. Biomedical papers of the Medical Faculty of the University Palacky, Olomouc, Czechoslovakia 158, 373-377

119. Schira, J., Gasis, M., Estrada, V., Hendricks, M., Schmitz, C., Trapp, T., Kruse, F., Kogler, G., Wernet, P., Hartung, H. P., and Muller, H. W. (2012) Significant clinical, neuropathological and behavioural recovery from acute spinal cord trauma by transplantation of a well-defined somatic stem cell from human umbilical cord blood. Brain : a journal of neurology 135, 431-446

120. Hartmann, I., Hollweck, T., Haffner, S., Krebs, M., Meiser, B., Reichart, B., and Eissner, G. (2010) Umbilical cord tissue-derived mesenchymal stem cells grow best under GMP-compliant culture conditions and maintain their phenotypic and functional properties. Journal of immunological methods 363, 80-89

121. Friedman, R., Betancur, M., Boissel, L., Tuncer, H., Cetrulo, C., and Klingemann, H. (2007) Umbilical cord mesenchymal stem cells: adjuvants for human cell transplantation. Biology of blood and marrow transplantation : journal of the American Society for Blood and Marrow Transplantation 13, 1477-1486

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of inhibiting a pathological immune response, comprising cell to cell contact and/or transfer of soluble materials in vivo, ex vivo, or in vitro from one or more fibroblasts to one or more antigen presenting cells, wherein the cell to cell contact and/or transfer of soluble materials from one or more fibroblasts to said antigen presenting cells reduces antigen presenting cell activity and/or reprograms said antigen presenting cells.

2. The method of claim 1, wherein the cell to cell contact and/or transfer of soluble materials from one or more fibroblasts to one or more antigen presenting cells occurs in vitro or ex vivo.

3. The method of claim 1 or 2, wherein following the cell to cell contact and/or transfer of soluble materials from one or more fibroblasts to one or more antigen presenting cells occurring in vitro or ex vivo, an effective amount of the fibroblasts are administered to an individual in need thereof.

4. The method of claim 1, wherein the cell to cell contact and/or transfer of soluble materials from one or more fibroblasts to one or more antigen presenting cells occurs in vivo, wherein the fibroblasts are administered to an individual.

5. The method of any one or claims 1-4, wherein the antigen presenting cell(s) is (are) selected from the group consisting of dendritic cells, B cells, innate lymphoid cells, and a combination thereof.

6. The method of claim 5, wherein the dendritic cells are selected from the group consisting of lymphoid dendritic cells, myeloid dendritic cells, myeloid suppressor cells, and a combination thereof.

7. The method of claim 5, wherein the innate lymphoid cells are selected from the group consisting of innate lymphoid cells (ILC)1, ILC2, ILC3, lymphoid tissue inducer cells, and a combination thereof.

8. The method of any one of claims 1-7, wherein the antigen presenting cell activity comprises, expression of MHC molecules on the surface of the antigen presenting cell, loading of antigen into MHC molecules, and/or expression of one or more costimulatory molecules on the antigen presenting cells.

9. The method of claim 8, wherein the costimulatory molecule(s) is (are) membrane-bound and/or soluble.

10. The method of claim 9, wherein the membrane-bound costimulatory molecules comprise at least one of CD40, CD80, CD86, or a combination thereof.

11. The method of claim 9 wherein the soluble costimulatory molecules comprise at least one of IL-12, IL-2, IL-11, IL-15, IL-18, or a combination thereof.

12. The method of any one of claims 1-11, wherein the fibroblasts are from tissue selected from the group consisting of placenta, cord blood, mobilized peripheral blood, omentum, hair follicle, skin, dermis, bone marrow, adipose tissue, Wharton’s Jelly, and a combination thereof.

13. The method of any one of claims 1-12, wherein the fibroblasts are pretreated with one or more toll like receptor (TLR) agonists.

14. The method of claim 13, wherein the fibroblasts are pretreated with TLR agonist(s) for a sufficient time and at a sufficient concentration to enhance immune modulatory activity.

15. The method of claim 14, wherein the immune modulatory activity comprises activity to suppress antigen presenting cell maturation and/or antigen presenting cell activity.

16. The method of any one of claims 13-15, wherein the TLR agonist(s) is (are) an selected from the group consisting of a TLR-1 agonist, TLR-2 agonist, TLR-3 agonist, TLR-4 agonist, TLR-5 agonist, TLR-6 agonist, TLR-7 agonist, TLR-8 agonist, TLR-9 agonist, and a combination thereof.

17. The method of claim 16, wherein the TLR-1 agonist comprises Pam3CSK4.

18. The method of claim 16, wherein the TLR-2 agonist comprises HKLM.

19. The method of claim 16, wherein the TLR-3 agonist comprises Poly:IC.

20. The method of claim 16, wherein the TLR-4 agonist is selected from the group consisting of LPS, buprenorphine, carbamazepine, fentanyl, levorphanol, methadone, cocaine, morphine, oxcarbazepine, oxycodone, pethidine, glucuronoxylomannan from Cryptococcus, morphine-3-glucuronide, lipoteichoic acid, β-defensin 2, small molecular weight hyaluronic acid, fibronectin EDA, snapin, tenascin C, and a combination thereof.

21. The method of claim 16, wherein the TLR-5 agonist comprises flagellin.

22. The method of claim 16, wherein the TLR-6 agonist comprises FSL-1.

23. The method of claim 16, wherein the TLR-7 agonist comprises imiquimod.

24. The method of claim 16, wherein the TLR-8 agonist comprises ssRNA40/LyoVec.

25. The method of claim 16, wherein the TLR-9 agonist comprises CpG oligonucleotide, ODN2006, agatolimod, or a combination thereof.

26. The method of any one of claims 3-25, wherein mesenchymal stem cells are administered with the fibroblasts.

27. The method of claim 26, wherein the mesenchymal stem cells enhance immune modulatory activity of the fibroblasts.

28. The method of claim 27, wherein the immune modulatory effects comprise suppression of maturation of antigen presenting cells.

29. The method of claim 27, wherein the immune modulatory effects comprise suppression of NF-kappa B activity and/or production, IL-2 production, IL-12 production, IL-15 production, IL-18 production, or a combination there of by the antigen presenting cells.

30. The method of any one of claims 3-28, wherein T regulatory cell production is concurrently increased with administration of the fibroblasts.

31. The method of claim 30, wherein the T regulatory cell production enhances a tolerogenic process and/or reprogramming of antigen presenting cells towards a tolerogenic phenotype.

32. The method of either claims 30 or 31, wherein the T regulatory cell production increase comprises the administration of a low dose of IL-2.

33. The method of claim 32, wherein the lose dose of IL-2 comprises 50,000 to 5,000,000 IU per day.

34. The method of claim 32, wherein the lose dose of IL-2 comprises 500,000 to 5,000,000 IU per day.

35. The method of claim 32, wherein the lose dose of IL-2 comprises 700,000 to 2,000,000 IU per day.

36. The method of claim 32, wherein the lose dose of IL-2 comprises 1,000,000 to 2,000,000 IU per day.

37. The method of claim 32, wherein the lose dose of IL-2 comprises 1,500,000 IU per day.

38. The method of any one of claims 1-37, wherein the pathological immune response comprises at least one autoimmune reaction, autoimmune disease, graft rejection, graft versus host disease, host versus graft disease, or a combination thereof.

39. The method of claim 2, wherein following the cell to cell contact and/or transfer of soluble materials from one or more fibroblasts to one or more antigen presenting cells occuring in vitro or ex vivo, an effective amount of the fibroblasts are administered to an individual having at least one autoimmune reaction, autoimmune disease, graft rejection, graft versus host disease, host versus graft disease, or a combination thereof.
